(19) United States
(12) Patent Application Publication
(10) Pub. No.: US 2021/0059551 A1
(43) Pub. Date: Mar. 4, 2021

(54) HIGH THROUGHPUT ECG HETEROGENEITY ASSESSMENT TO DETERMINE PRESENCE OF CORONARY ARTERY STENOSIS

(71) Applicants: Bruce D. Nearing, North Reading, MA (US); Richard L. Verrier, Wellesley Hills, MA (US); BETH ISREAL DEACONESS MEDICAL CENTER, INC., Boston, MA (US)

(72) Inventors: Bruce D. NEARING, North Reading, MA (US); Richard L. VERRIER, Wellesley Hills, MA (US)

(73) Assignee: Beth Israel Deaconess Medical Center,, Boston, MA (US)

(21) Appl. No.: 16/979,390

(22) PCT Filed: Mar. 8, 2019
(86) PCT No.: PCT/US2019/021344

§ 371 Date: Sep. 9, 2020

Related U.S. Application Data
(60) Provisional application No. 62/640,970, filed on Mar. 9, 2018.

(51) Int. Cl. A61B 5/0452; A61B 5/0428; A61B 5/02; A61B 5/00; G16H 50/20; G16H 50/30

(52) U.S. Cl. CPC A61B 5/0452; A61B 5/04286; A61B 5/02007; A61B 5/044; A61B 5/4884; G16H 50/20; G16H 50/30; A61B 5/7246

(57) ABSTRACT

A method and system for high-throughput detection of coronary artery stenosis observes trends in abnormal or pathologic morphology of the electrocardiogram (ECG). A first set of ECG signals is monitored from a patient. A baseline measurement is generated from the monitored first set of ECG signals to contain nonpathologic ECG morphologies in each lead. A second set of ECG signals is monitored from the patient and a second mean measurement during or after stress is generated from the second set of ECG signals. A residuum signal is generated for each lead based on the baseline measurement and the second mean measurement. The residuum signals are averaged across the leads for each timepoint. T-wave heterogeneity is quantified based on the generated residuum signals and the averaged residuum signal at each timepoint, and used to detect coronary artery stenosis.

### BACKGROUND

### Field

Embodiments herein relate to systems and methods for determining potential health risks by analyzing electrocardiograms (ECG).

### Background

There is mounting evidence that symptomatic diabetic patients are at elevated risk for cardiovascular mortality prior to angiographically demonstrable progression to obstructive coronary artery disease (CAD). Several factors appear to be responsible, including the presence of diffuse coronary atherosclerosis and microvascular and diastolic dysfunction. An important exacerbating factor is the coexistence of diabetic autonomic neuropathy, which impacts on the vasodilatory response of coronary resistance vessels due to increased sympathetic tone and cardiac arrhythmias.

Noninvasive detection of coronary artery stenosis of large epicardial vessels remains a daily challenge in contemporary cardiology. The two main first-line diagnostic techniques are exercise tolerance testing (ETT) and pharmacological stress testing along with symptom evaluation. Each test is conducted either independently or in conjunction with echocardiography or nuclear imaging. The induction of ETT-induced ST-segment depression is the most widely employed ECG sign of coronary artery disease (CAD)-associated myocardial ischemia.

Notwithstanding an extensive experience, it is generally recognized that these tests in their current form yield excess numbers of both false positive and false negative tests, as assessed by the “gold standard” of diagnostic coronary angiography. Several other ECG parameters beyond ST-segment have been evaluated to improve the diagnostic yield of stress testing, including ST/heart rate slope or index and ST/heart rate recovery loop. However, none has been shown to provide convincing diagnostic value in clinical practice. ETT-based detection of CAD using ST-segment measurement is particularly problematic in women due to the excess false positive rate, which ranges from 25-50%.

### BRIEF SUMMARY

Example methods and systems are described herein for embodying a high-throughput approach to isolating abnormal ECG signals to capture and measure morphologic ECG changes that may be associated with ventricular tachycardia, nonflow-limiting coronary artery stenosis, or flow-limiting coronary artery stenosis.

In an embodiment, an example method is described. The method includes receiving a first set of electrocardiogram (ECG) signals from spatially separated leads; generating a median beat signal associated with the morphology of each ECG signal of the first set of ECG signals; receiving a second set of ECG signals from spatially separated leads; generating a second median beat signal associated with the morphology of each ECG signal of the second set of ECG signals; calculating, for each lead, a residuum signal based on the first and second median beat signals; averaging the residuum signals across the leads to produce an averaged residuum signal; and quantifying ECG characteristics based on the residuum signals and the averaged residuum signal. The quantified ECG characteristics are used to detect coronary artery stenosis. For example, T-wave heterogeneity may be quantified based on this method and used to determine the presence of coronary artery stenosis, including the relative location of the blockage (right side or left side of the heart). This method may also be used to quantify P-wave changes indicative of risk of atrial arrhythmias or ST-segment changes among spatially separated leads to identify regions of myocardial ischemia.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The patent or application file contains at least one drawing executed in color. Copies of this patent or patent application publication with color drawing(s) will be provided by the Office upon request and payment of the necessary fee.

The accompanying drawings, which are incorporated herein and form part of the specification, illustrate the present invention and, together with the description, further serve to explain the principles of the present invention and to enable a person skilled in the relevant art(s) to make and use the present invention.

FIG. 1 illustrates leads of an ECG device placed on a patient, according to an embodiment.

FIG. 2 illustrates signal processing techniques of an ECG signal, according to an embodiment.

FIG. 3 illustrates results of calculating R-wave heterogeneity in simulated ECGs, according to an embodiment.

FIG. 4 illustrates results of calculating T-wave heterogeneity in simulated ECGs, according to an embodiment.

FIG. 5 illustrates results of measured R-wave heterogeneity before a ventricular tachycardia event, according to an embodiment.

FIG. 6 illustrates results of measured T-wave heterogeneity before a ventricular tachycardia event, according to an embodiment.

FIG. 7 illustrates results of measured R-wave and T-wave heterogeneity before a ventricular tachycardia event, according to an embodiment.

FIG. 8 illustrates results of measured atrial ECG heterogeneity before onset of atrial fibrillation, according to an embodiment.

FIG. 9 illustrates an example ECG system, according to an embodiment.

FIG. 10 illustrates an example method, according to an embodiment.

FIG. 11 illustrates signal processing techniques of an ECG signal, according to an embodiment.

FIG. 12 illustrates an example method, according to an embodiment.

FIG. 13 illustrates digitized ECG tracings of T-wave heterogeneity as interlead splay in repolarization morphology during rest and exercise in a representative control subject (upper panels) and representative case (lower panels), according to an embodiment.

FIG. 14 demonstrates the absence of change in T-wave heterogeneity (TWH) from rest to exercise in control subjects (open bars, n=9) compared to an increase in TWH in cases (filled bars, n=20), according to an embodiment.

FIG. 15 illustrates the change in T-wave heterogeneity (TWH) from rest to exercise in control subjects and in cases, according to an embodiment.

FIG. 16 illustrates a comparison by quartiles of range of T-wave heterogeneity (TWH) levels in control subjects (n=9) and in cases (n=20) during exercise, according to an embodiment.

FIG. 17 illustrates digitized ECG tracings of T-wave heterogeneity (TWH) as interlead splay in repolarization morphology during rest and exercise tolerance testing (ETT) in a representative control subject (upper panels) and a representative case (lower panels), according to an embodiment.

FIG. 18 illustrates TWH levels measured in microvolts for cases and control subjects at rest and under stress, according to an embodiment.

FIG. 19 illustrates area under the receiver-operator curves (AUCs) for TWH increase under exercise induced stress (e.g., treadmill) and pharmacological induced stress (e.g., Dipyridamole), according to an embodiment.

FIG. 20 illustrates an area under the receiver-operator curve (AUC) subset analysis for males, females, and diabetic and nondiabetic patients, according to an embodiment.

FIG. 21 illustrates digitized ECG tracings of T-wave heterogeneity (TWH) as interlead splay in repolarization morphology of superimposed simultaneous ECGs during rest and exercise tolerance testing (ETT) in a representative control subject (upper panels) and a representative case (lower panels), according to an embodiment.

FIG. 22 illustrates digitized ECG tracings of T-wave heterogeneity (TWH) as interlead splay in repolarization morphology of superimposed simultaneous ECGs during rest and IV dipyridamole testing in a representative control subject (upper panels) and a representative case (lower panels), according to an embodiment.

FIG. 23 illustrates TWH_{V4-6} levels measured in microvolts for cases and controls at rest and ETT and dipyridamole testing, according to an embodiment.

FIG. 24 illustrates area under the receiver-operator curve (AUC) for any flow-limiting coronary artery stenosis for ETT (upper panel) and for dipyridamole (lower panel), according to an embodiment.

FIG. 25 illustrates TWH_{V1-3} levels measured in microvolts for cases and controls at rest and ETT testing and dipyridamole testing, according to an embodiment.

FIG. 26 illustrates area under the receiver-operator curve (AUC) for cases and controls at rest and ETT testing and dipyridamole testing, according to an embodiment.

FIG. 27 illustrates digitized ECG tracings of T-wave heterogeneity (TWH) as interlead splay in repolarization morphology of superimposed simultaneous ECGs during rest and regadenoson stress testing in a representative control subject (upper panels) and a representative case (lower panels), according to an embodiment.

FIG. 28 illustrates TWH levels measured in microvolts for cases and controls at rest and regadenoson stress testing, according to an embodiment.

FIG. 29 illustrates a box plot of TWH by quartiles in cases and controls in response to regadenoson, according to an embodiment.

FIG. 30 illustrates area under the receiver-operating characteristic curve (AUC) for TWH to identify for flow-limiting coronary artery stenosis at peak stress, according to an embodiment.

FIG. 31 illustrates TWH's capacity to identify flow-limiting coronary artery stenosis at peak stress in women (left) was similarity to that in men (right) based upon areas under the receiver-operating characteristic curve (AUC), according to an embodiment.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

This specification discloses one or more embodiments that incorporate the features of this invention. The disclosed embodiment(s) merely exemplify the present invention. The scope of the present invention is not limited to the disclosed embodiment(s). The present invention is defined by the claims appended hereto.

The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Embodiments of the present invention may be implemented in hardware, firmware, software, or any combination thereof. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

Before describing such embodiments in more detail, however, it is instructive to present an example environment in which embodiments of the present invention may be implemented.

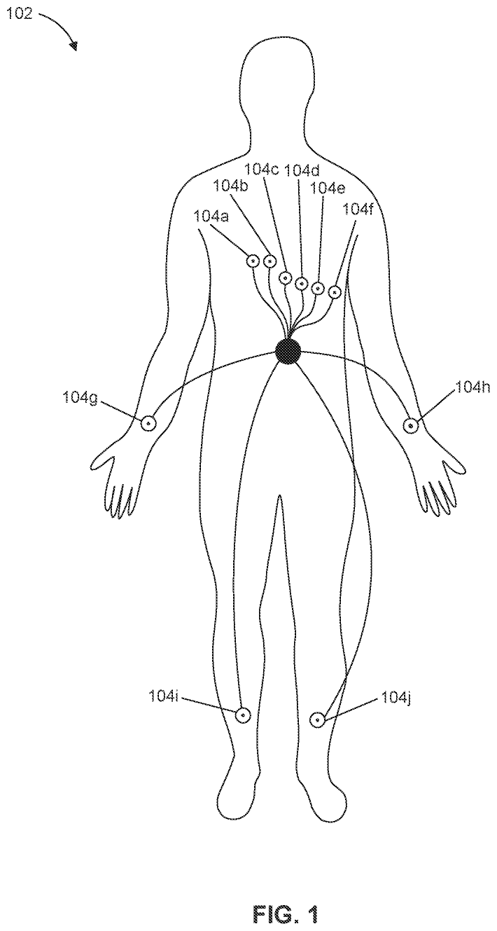

FIG. 1 illustrates a patient 102 that is attached to various leads of an ECG recording device, according to an embodiment. The leads may be used to monitor a standard 12-lead ECG. In this example, six leads (leads 104a-f) may be placed across the chest of patient 102 while four other leads (leads 104g-j) are placed with two near the wrists and two near the ankles of patient 102.

It should be understood that the exact placement of the leads is not intended to be limiting. For example, the two lower leads 104i and 104j may be placed higher on the body, such as on the outer thighs. In another example, leads 104g and 104h are placed closer to the shoulders while leads 104i and 104j are placed closer to the hips of patient 102. In still other examples, not all ten leads are required to be used in order to monitor ECG signals from patient 102.

In an embodiment, signals are monitored from each of leads 104a-j during a standard 12-lead ECG recording. The resulting ECG signal may be analyzed over time to determine various health factors such as heart rate, strength of heart beat, and any indicators of abnormalities. However, changes in the various signals received amongst leads 104a-j may be very small and difficult to detect. Any trend in the changing signal amplitude for certain areas of the ECG morphology could be vital in predicting the onset of potentially fatal heart complications. For example, prediction of heart arrhythmias, or the presence of coronary artery stenosis, may be possible by observing trends in the R-wave heterogeneity, T-wave heterogeneity, P-wave heterogeneity and/or T-wave alternans from the monitored ECG signals. The observation of using T-wave alternans as a predictor for heart arrhythmias has been discussed previously in U.S. Pat. No. 6,169,919, the disclosure of which is incorporated by reference herein in its entirety. Spatial differences in ST-segment morphology, termed ST-segment heterogeneity, may provide evidence of regionality of myocardial ischemia, a characteristic that contributes to risk for lethal arrhythmia.

The challenge is to separate these biologically significant microvolt-level changes from the intrinsic differences in ECG morphology. In an embodiment, the technique employed herein utilizes a multi-lead ECG median-beat baseline for each lead, which allows for the determination of ECG residua by subtraction of the baseline from the collected ECG signals. These residua may be evaluated in association with R-wave and T-wave heterogeneity analysis and other parameters for heart arrhythmia prediction, myocardial ischemia assessment, or determination of coronary artery stenosis. Ultimately, the implementation of embodiments described herein can lead to improved identification of individuals at risk for lethal heart complications and sudden cardiac death and can serve as a guide to therapy.

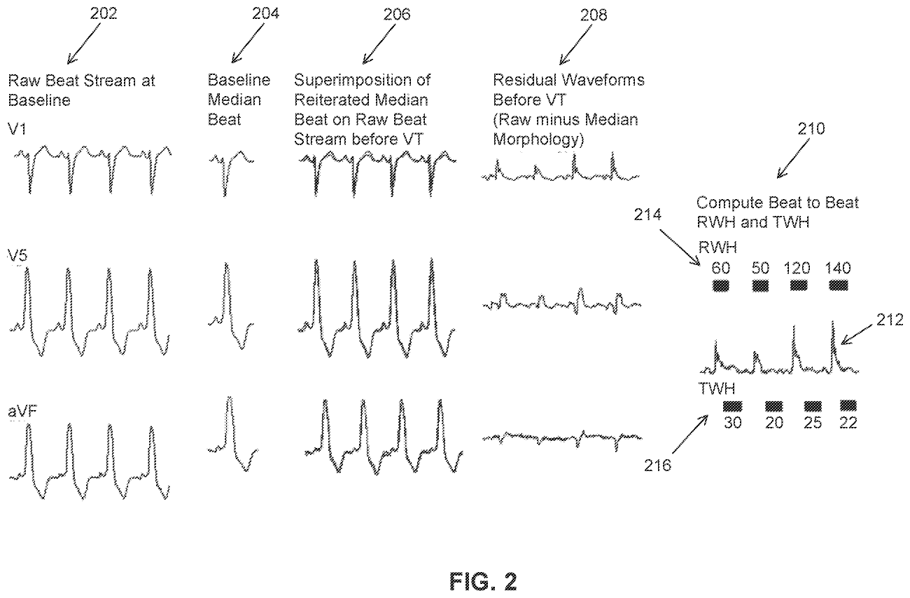

FIG. 2 illustrates a signal processing procedure for generating ECG residua and detecting changes, for example, in R-wave and T-wave heterogeneity from the signals received from various leads, according to an embodiment. For simplicity, the signal processing procedure described with reference to FIG. 2 will be referred to herein as the multi-lead residuum procedure. In one example, signals from three different ECG leads (V1, V5, and aVF) are shown in column 202. It should be understood that signals from any number of leads may be used. The ECG signals to be analyzed in accordance with the present disclosure may be sensed in real-time from a patient and processed on a real-time or near real-time basis (e.g., within seconds or minutes of being collected from a patient). Alternatively, the ECG signals may be received from some storage medium (e.g., an analog or digital storage device) for analysis in accordance with the present disclosure.

A baseline recording 202 is generated from the signals received from each of the ECG leads, according to an embodiment. In one example, the baseline measurement is generated by computing a median-beat 204 from the collected signals shown in column 202. An example calculation of the median-beat B_{i,n}(t) for n=1 . . . N beats, where i=1 . . . M ECG signals and M=all ECG leads, is shown below in equation 1.

B_{i,n}(t)=B_{i,n-1}(t)+Δ_{i,n} (1)
Δ_{i,n}=−32 if δ≤−32
Δ_{i,n}=δ if −1≥δ≥−32
Δ_{i,n}=−1 if 0≥δ≥−1
Δ_{i,n}=0 if δ=0
Δ_{i,n}=1 if 1≥δ≥0
Δ_{i,n}=δ if 0≥δ≥1
Δ_{i,n}=32 if δ≤32
where δ=(ECG_{i,n-1}(t)−B_{i,n-1}(t))/8
and B_{i,0}(t)=ECG_{i,0}(t)
i=1 . . . M ECG signals
n=1 . . . N Baseline Beats

In an embodiment, the sequence starts with the first beat, and each successive beat then contributes a limited amount to the median-beat computation in each ECG lead. The baseline measurement contains nonpathologic morphologies in each ECG lead and may be associated with a period of quiet rest when morphology differences over time are at a minimum. This baseline measurement may be calculated by computing the median beat 204 over a time period between, for example, 5 and 10 minutes. Collection times over 10 minutes may be used as well, but would typically not be necessary for calculating a stable baseline signal. Alternatives to the use of median beats include calculating the baseline signal from an average of all the beats in the baseline time period or using a single, representative beat from the baseline time period as the baseline signal. These methods are simpler but not as robust as median beat calculation. Baseline measurements of the ECG signals received via leads V1, V5, and aVF are shown in column 204.

Once the baseline measurement 204 has been generated, a second set of ECG recordings, ECGᵢ(t), is made. In an embodiment, the second set of ECG recordings is made soon after (e.g., immediately after) the baseline recording. However, it is also possible that the second set of ECG recordings is made at any period of time after the baseline recording has been generated. For example, the baseline recording for a particular patient may be saved and used a year later when that patient returns to have a second set of ECG recordings made. It should also be understood that there is no restriction as to the duration of the second set of ECG recordings.

In an embodiment, the baseline measurement B_{i,N}(t) and the second set of ECG recordings ECGᵢ(t) for each lead are used to generate a residuum signal for each lead. In one example, each baseline measurement beat is reiterated and aligned either temporally or spatially with the various beats from the second ECG recordings for each lead in order to subtract the morphologies from one another (e.g., for a particular lead, the baseline measurement beat is subtracted from the various beats of the second ECG recording). In another example, each baseline measurement beat is reiterated and aligned either temporally or spatially with the various beats from the second ECG recordings for each lead, and the residuum signal for each lead is calculated as a quotient on a point by point basis where the numerator represents the second ECG recording and the denominator represents the baseline measurement. The residuum signal may represent a difference when subtracting, while the residuum signal may represent a fractional change when dividing.

Column 206 illustrates the superimposition of the baseline measurement 204 B_{i,N}(t) over the second set of ECG recordings ECGᵢ(t) in order to subtract the baseline signal, according to one embodiment. The residuum signal resulting from the subtraction for each lead is illustrated in column 208. Likewise, equation 2 below provides the generation of the residuum signal eᵢ(t) when subtracting.

eᵢ(t)=ECGᵢ(t)−B_{N}(t) (2)
i=1 . . . M ECG signals
N=Number of beats in baseline sequence

According to another embodiment, a median beat is also calculated for the second set of ECG recordings, ECGᵢ(t) to produce a second median beat for each lead. The median baseline beat for each lead may then be subtracted from the second median beat for each lead to generate a residuum signal for each lead. This could be done as an alternative to the superimposition of the baseline measurement 204 over the second set of ECG recordings, ECGᵢ(t), illustrated in Column 206. In this alternate embodiment, the median baseline beat for each lead would be superimposed over the second median beat for each lead to generate the residuum signal for each lead.

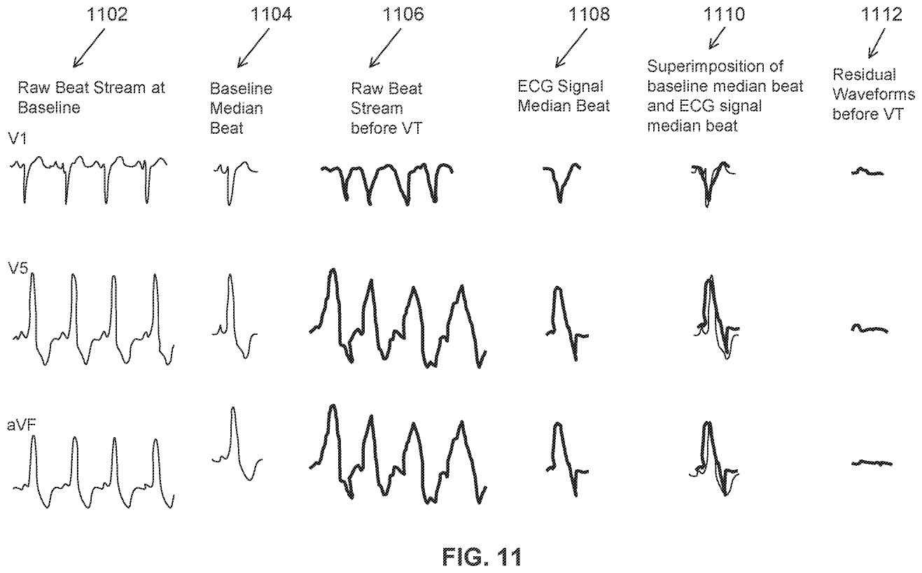

An example of this embodiment using a second median beat for each lead is illustrated in FIG. 11. Many of the features in FIG. 11 are similar to those already discussed with reference to FIG. 2 above. For example, a baseline recording 1102 is generated from the signals received from each of the ECG leads V1, V5, and aVF. As noted before, any number of leads may be used. A baseline median beat 1104 is calculated for each lead according to Equation 1 above. A second set of ECG signals are collected across the leads V1, V5 and aVF as illustrated in column 1106.

Column 1108 illustrates the generation of a median beat for the second set of ECG signals (i.e., a second median beat) for each lead, according to an embodiment. The calculation of this second median beat may be substantially similar to calculation of the baseline median beat illustrated in column 1104. For example, the amplitude of the second set of ECG signals as a function of time may be given by S_{i,m}(t) for m=1 . . . M beats and i=1 . . . I ECG signals, where I=all ECG leads. The measurement signal S_{i,m}(t) may be obtained, for example, from a 10 second ECG segment, or a short ECG segment during an exercise stress test or Holter recording. An example calculation of the ECG signal median-beat is shown below in equation 3.

S_{i,m}(t)=S_{i,m-1}(t)+Δ_{i,m} (3)
Δ_{i,m}=−32 if δ≤−32
Δ_{i,m}=δ if −1≥δ≥−32
Δ_{i,m}=−1 if 0≥δ≥−32
Δ_{i,m}=0 if δ=0
Δ_{i,m}=1 if 1≥δ≥0
Δ_{i,m}=δ if 0≥δ≥1
Δ_{i,m}=32 if δ≤32
where δ=(ECG_{i,m-1}(t)−S_{i,m-1}(t))/8
and S_{i,0}(t)=ECG_{i,0}(t)
i=1 . . . I ECG signals
m=1 . . . M Baseline Beats
t=−P . . . +R
where t=−P is the time of the P-Wave Onset
where t=0 is the time of the R-Wave Peak
where t=+R is the time of the T-Wave End

Once both a baseline median beat and a second median beat have been calculated for each lead, the median beats may be superimposed so that R-waves are aligned. An example of this superimposition is illustrated in column 1110 of FIG. 11. In an embodiment, the baseline median beat is subtracted from the second median beat to generate a residuum signal for each lead as illustrated in column 1112. In another example, the residuum signal for each lead is calculated as a quotient on a point by point basis where the numerator represents the second median beat and the denominator represents the baseline median beat. Likewise, equation 4 below provides the generation of the residuum signal eᵢ(t) when subtracting.

eᵢ(t)=S_{i,M}(t)−B_{i,N}(t) (4)
i=1 . . . I ECG signals
N=Number of beats in Baseline sequence
M=Number of beats in Measurement sequence
t=−P . . . +R
where t=−P is the time of the P-Wave Onset
where t=0 is the time of the R-Wave Peak
where t=+R is the time of the T-Wave End

Once the residuum signals have been calculated for each lead using any of the embodiments described above, they may be used for calculating the R-wave heterogeneity (RWH) and T-wave heterogeneity (TWH), according to an embodiment. By observing trends in the RWH and/or TWH, cardiac events such as ventricular tachycardia may be predicted well in advance, allowing for preventive procedures to be taken. The RWH and TWH may be calculated by first averaging the spatio-temporal signals of each of the residuum signals to generate an averaged residuum signal as shown below in equation 5.

$$\overline{e ( t )} = \frac{1}{M} {{\sum }_{i = 1}}^{M} {e}_{i} ( t ) \qquad ( 5 )$$

In the above equation, and for other equations used herein, M is an integer greater than two and equal to the number of total ECG signals collected. In one example, one ECG signal is recorded from each lead of the standard 12-lead ECG.

Next, in an embodiment, a second central moment 212 about the averaged residuum signal is determined by taking the mean-square deviation of the various ECG signals about the average signal. This step is shown below in Equation 6.

$${μ}_{2} ( t ) = \frac{1}{M} {{\sum }_{i = 1}}^{M} {( {e}_{i} ( t ) - \overline{e ( t )} )}^{2} \qquad ( 6 )$$

With the second central moment 212 calculated, RWH 214 may be determined as the maximum square root of the second central moment of the ECG residua occurring within the QRS segment. In an embodiment, the QRS segment begins at the Q-wave and ends at the J-point of a standard ECG signal. Equation 7 below provides an example calculation for the RWH.

$$RWH = {MAX}_{Q - Waveonset \leq t \leq J - point} \sqrt{{μ}_{2} ( t )} \qquad ( 7 )$$

TWH 216 may be determined as the maximum square root of the second central moment of the ECG residua occurring within the JT interval. The JT interval occurs approximately from 60 to 290 msec after the R-wave of a standard ECG signal. Equation 8 below provides an example calculation for the TWH.

$$TWH = {MAX}_{J - point \leq t \leq T - waveend} \sqrt{{μ}_{2} ( t )} \qquad ( 8 )$$

Computation of residuum signals may be also useful in calculating heterogeneity of the P-Wave (PWH) from its onset to offset, which relates to atrial arrhythmias, and heterogeneity of the ST-Segment (STWH) from the J-point to the onset of the T-wave, which identifies nonhomogeneous features of myocardial ischemia.

$$\begin{matrix}PWH = {MAX}_{P - Waveonset \leq t \leq P - Waveoffset} \sqrt{{μ}_{2} ( t )} & ( 9 ) \\ STWH = {MAX}_{J - point \leq t \leq T - Waveonset} \sqrt{{μ}_{2} ( t )} & ( 10 )\end{matrix}$$

Column 210 illustrates results 212 of second central moment analysis of the residuum signals as well as the areas of the signal that correspond to RWH measurements 214 and TWH measurements 216, according to an embodiment. As shown in the example, the RWH and TWH measurements may change between beats. Peak levels of RWH and TWH are averaged for each 15-sec sampling period. Trends in the changing RWH and/or TWH may be used to identify short- or long-term risk for cardiac arrhythmias. In one example, the RWH and/or TWH may be reported over a given period of time for further analysis and/or data presentation.

Using both the baseline median beat and second median beat in the calculation of a residuum signal for each lead allows for high-throughput analysis of a plurality of patients. In one example study, over 5600 patient ECGs from a database were analyzed with a processing time of a few seconds per patient to yield highly predictive results in terms of assessing risk for cardiovascular mortality and sudden cardiac death (SCD). The patients [5618 adults, 46% men; age 50.9±0.2 years (means±SEM)], were enrolled in the Health 2000 Survey, an epidemiological survey representative of the entire Finnish adult population. During follow-up of 7.7±0.2 years, a total of 72 SCDs occurred. Increased RWH, JWH and TWH in left precordial leads (V4-V6) were univariately associated with SCD (P<0.001, each). When adjusted with clinical risk markers, JWH and TWH remained independent predictors of SCD. Increased TWH (≥102 μV) was associated with a 1.7-fold adjusted relative risk (95% confidence interval [CI]: 1.0-2.9; P=0.048) and increased JWH (≥123 μV) with a 2.0-fold adjusted relative risk for SCD (95% CI: 1.2-3.3; P=0.006). When both TWH and JWH were above threshold, the adjusted relative risk for SCD was 2.9-fold (95% CI: 1.5-5.7; P=0.002). When all heterogeneity measures (RWH, JWH and TWH) were above threshold, the risk for SCD was 3.2-fold (95% CI: 1.4-7.1; P=0.009).

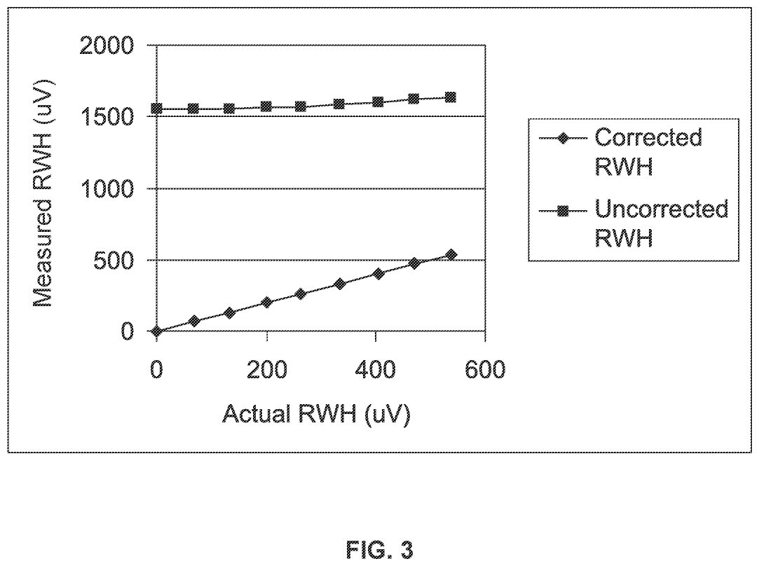

FIG. 3 illustrates results for measuring RWH in simulated ECG signals with various RWH levels. The ECG signals were generated using a C++ program with P-waves, R-waves, T-waves, and ST segments approximated by geometric shapes whose relative timing and amplitude were similar to surface ECGs. The results in FIG. 3 demonstrate that the measured RWH (y-axis) was highly correlated with the actual input RWH (x-axis) when corrected by using the multi-lead residuum procedure (diamonds). However, when uncorrected, the program was unable to determine accurately the RWH as shown by the uncorrected data points (squares), as results varied by up to 1500 microvolts from the input RWH signal.

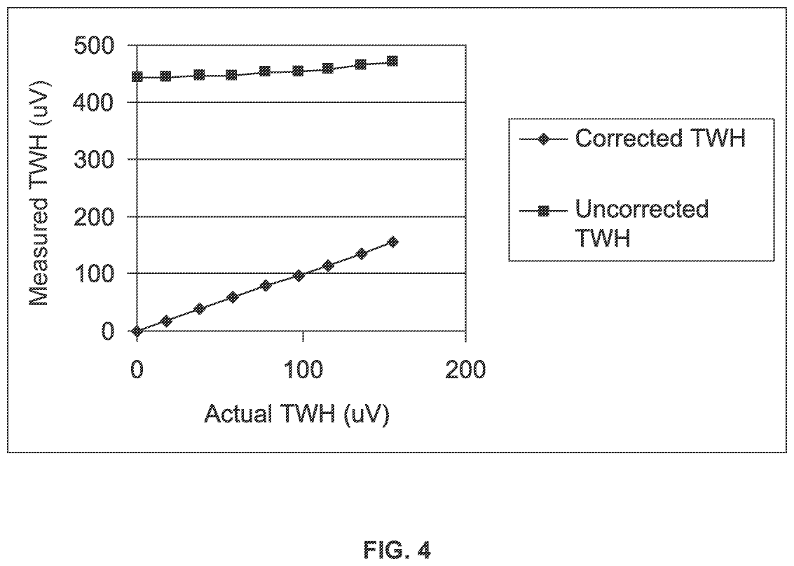

FIG. 4 illustrates results for measuring TWH in simulated ECG signals with various TWH levels. The ECG signals were generated using a C++ program with P-waves, R-waves, T-waves, and ST segments approximated by geometric shapes whose relative timing and amplitude were similar to surface ECGs. The results in FIG. 4 demonstrate that the measured TWH (y-axis) was highly correlated with the actual input TWH (x-axis) when corrected by using the multi-lead residuum procedure (diamonds). However, when uncorrected, the program was unable to determine accurately the TWH as shown by the uncorrected data points (squares), as results varied by up to 450 microvolts from the input TWH signal.

Thus, the RWH and TWH algorithm accurately tracked heterogeneities in R-wave and T-wave morphology in simulated ECGs when using the multi-lead residuum procedure but not in its absence. When calculating the residua, a linear relationship between the input and output values of RWH (range: 0-538 μV) and TWH (0-156 μV) estimated by second central moment analysis with a correlation coefficient of r²=0.999 (P<0.001) was observed.

The embodied multi-lead residuum procedure for accurately determining RWH and TWH was validated via the simulation experiments shown in FIGS. 3 and 4. However, analysis of ECGs from a clinical trial was also conducted to demonstrate the capacity of the procedure to predict dangerous cardiac complications such as ventricular tachycardia.

The capacity of multi-lead ECG residua to predict ventricular arrhythmia was examined by comparing RWH and TWH output with and without calculation of the residua in clinical ambulatory ECG recordings obtained in hospitalized patients with non-sustained ventricular tachycardia. The PRECEDENT (Prospective Randomized Evaluation of Cardiac Ectopy with Dobutamine or Nesiritide Therapy) trial (www.clinicaltrials.org #NCT00270400) enrolled 255 patients aged ≥18 years with NYHA class III or IV congestive heart failure and symptomatic, decompensated congestive heart failure for which inpatient, single-agent, intravenous therapy with either nesiritide or dobutamine was deemed appropriate. All patients were monitored by ambulatory ECG recording for the 24-hour period immediately before the start of the study drug (pre-randomization ambulatory ECG tape).

Ambulatory ECGs recorded during the pre-randomization phase of the PRECEDENT trial were analyzed from all 22 patients who experienced a single bout of ventricular tachycardia (≥4 beats at heart rates of >100 beats/min) following 120 minutes of stable sinus rhythm and without atrial fibrillation. The Beth Israel Deaconess Medical Center Committee on Clinical Investigations certified the exempt status of this reanalysis of existing data from a completed clinical trial under exemption number 4 of the Code of Federal Regulations, 45 CFR 46.101(b).

The continuous ECGs were analyzed with and without correction by ECG residua in leads V1, V5, and aVF by subtracting the median-beat baseline ECG, which was generated from ECGs recorded during a quiescent period at 60 to 75 minutes before the arrhythmia occurred. Then, the ECG heterogeneity signal was computed from the ECG residua as the square root of the sum of the squares of the differences between the corrected signal and the mean of the corrected signals. RWH was calculated as the maximum value of the heterogeneity signal in the interval from the beginning of the Q wave to the end of the S wave. TWH was calculated as the maximum value of the heterogeneity signal in the interval between the J point and the end of the T wave. The analysis window began at 75 minutes before ventricular tachycardia. RWH and TWH maxima were computed for each 15-second interval, comparing signals in leads V1, V5, and aVF, and averaged over 15-minute epochs. Correlation coefficients of input-output relationships were calculated for input-output relationships by Pearson's coefficient. RWH and TWH levels at 45-60, 30-45, 15-30, and 0-15 minutes were compared with baseline at 60 to 75 minutes before the onset of the arrhythmia in PRECEDENT trial patients. ANOVA was used with Tukey test for multiple comparisons (*p<0.05).

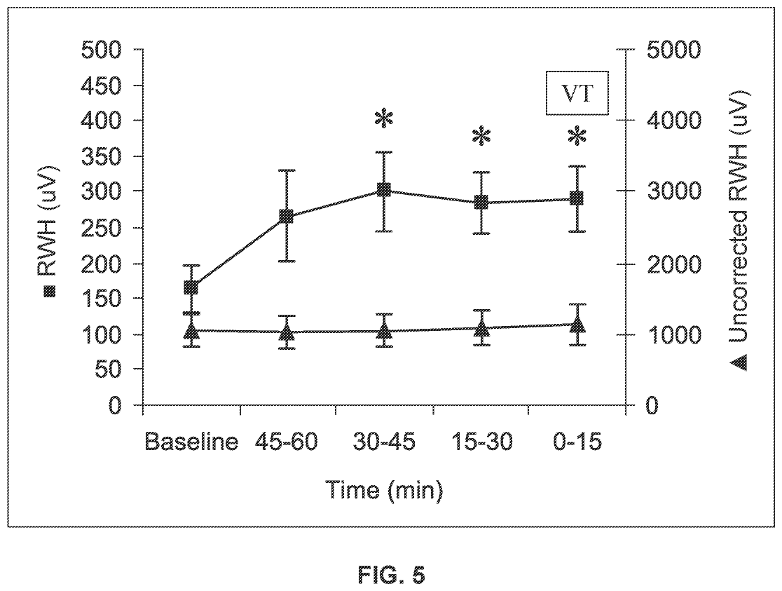

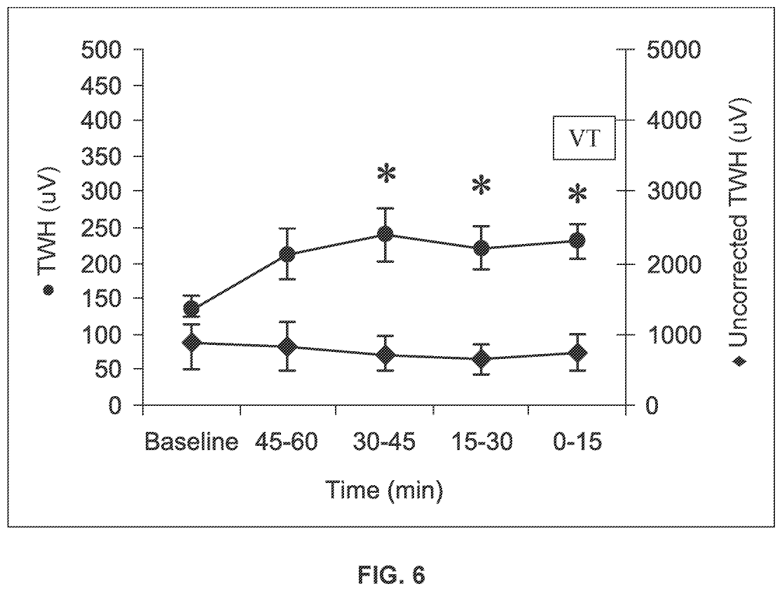

FIGS. 5 and 6 illustrate the results for the RWH and TWH respectively obtained for those patients prior to ventricular tachycardia. A noticeable crescendo in RWH (FIG. 5) and TWH levels (FIG. 6) was observed prior to ventricular tachycardia when using the multi-lead residuum procedure (left y-axes). Maximum RWH across leads V1, V5, and aVF rose from 164.1±33.1 μV at baseline to 299.8±54.5 μV at 30 to 45 minutes before the arrhythmia (P<0.05). Meanwhile, maximum TWH across leads V1, V5, and aVF rose from 134.5±20.6 μV at baseline to 239.2±37.0 μV at 30 to 45 minutes before the arrhythmia (p<0.05). Just before ventricular tachycardia, maximum RWH and TWH levels remained elevated at 289.5±45.9 and 230.9±24.7 μV, respectively (p<0.05). Although the extent of change varied among patients, the crescendo pattern in ECG heterogeneity before non-sustained ventricular tachycardia was consistent (Pearson correlation coefficient comparing RWH and TWH, 0.51; P=0.01). In 20 of 22 (91%) patients, RWH or TWH remained elevated before onset of non-sustained ventricular tachycardia.

When R-wave and T-wave heterogeneity were calculated without employing the multi-lead residuum procedure, the levels of both RWH (FIG. 5) and TWH (FIG. 6) were high during the initial baseline period (right y-axes). The values were 1061.0±222.9 μV for RWH and 882.5±375.2 μV for TWH and were not statistically different at the time of onset of ventricular tachycardia.

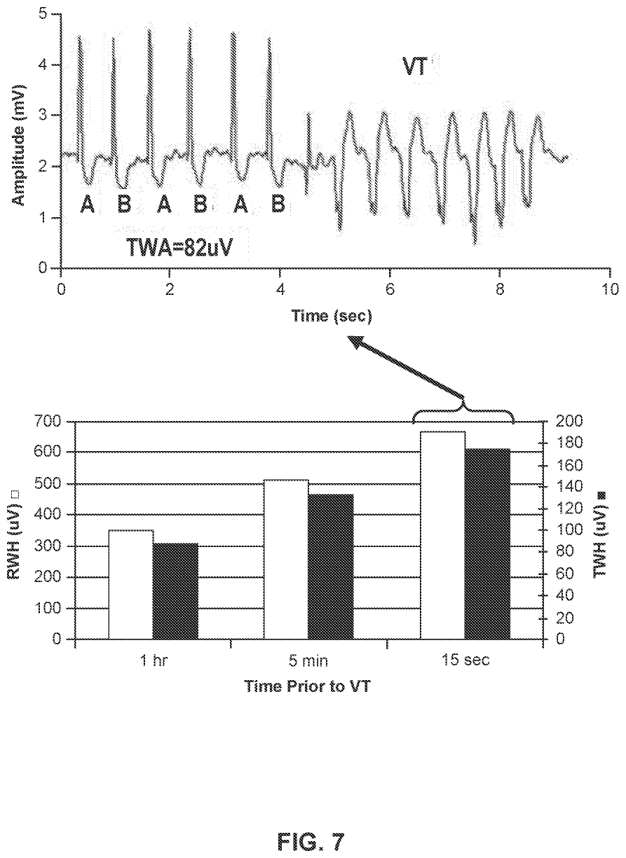

T-wave alternans (TWA) is another indicator of risk for lethal cardiac arrhythmias and can also be measured from the ECG along with the TWH measurements, according to an embodiment. FIG. 7 (lower panel) provides an example of the measured TWH (right y-axis) and RWH (left y-axis) of one patient at various times before the patient experienced ventricular tachycardia. Also illustrated is the measured TWA (˜82 μV) (upper panel) during the time leading up to the ventricular tachycardia. This patient exhibited increased levels of RWH and TWH that heralded the onset of TWA and ventricular tachycardia.

As mentioned previously, PWH reflects the depolarization phase of the atria. An intra-cardiac lead may be used to measure both atrial depolarization and repolarization heterogeneity more accurately. The latter reflects the repolarization phase of the atria. Typically, the repolarization phase of the atria is difficult to detect using surface leads as it is masked by the large R-wave deflection, which reflects ventricular depolarization. The intra-cardiac lead is less susceptible to noise and is capable of measuring the atrial repolarization heterogeneity. In an embodiment, both the repolarization and depolarization phases of the atria are used to determine the full atrial ECG heterogeneity.

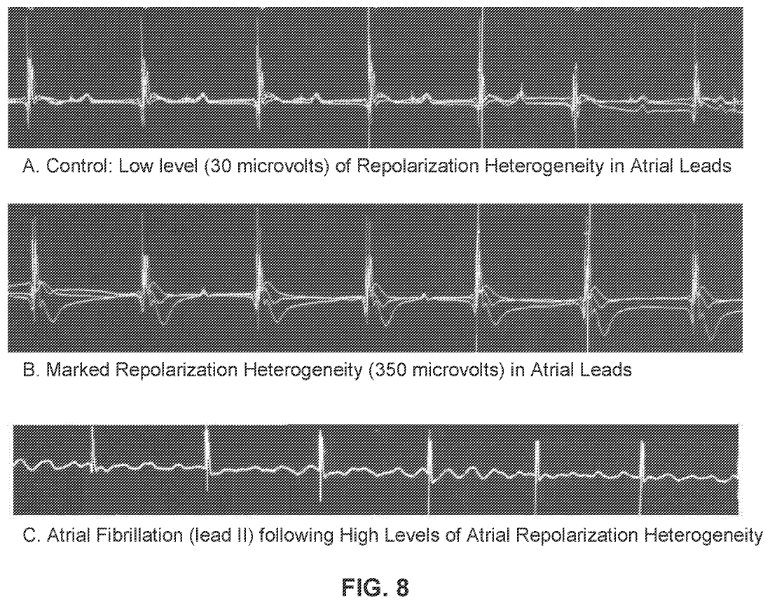

FIG. 8 illustrates results of measured atrial ECG heterogeneity before onset of atrial fibrillation, according to an embodiment. The recordings are of atrial ECGs prior to and during vagus nerve stimulation in a porcine model. This procedure replicates a condition of heightened vagus nerve activity, which is an important factor known to predispose to atrial fibrillation in patients. Prior to vagus nerve stimulation (panel A), ECG signals recorded from three pairs of electrodes on an intra-cardiac catheter show that the waveforms are relatively superimposable. In another embodiment, as few as two pairs of electrodes on an intra-cardiac catheter may be used to record the atrial ECGs. During vagus nerve stimulation (panel B), there is a marked splay in the repolarization phase of the atrial ECG. Shortly thereafter (panel C), atrial fibrillation developed, as indicated by a chaotic, irregular pattern in the isoelectric phase between the distinct R-wave spikes in the ECG.

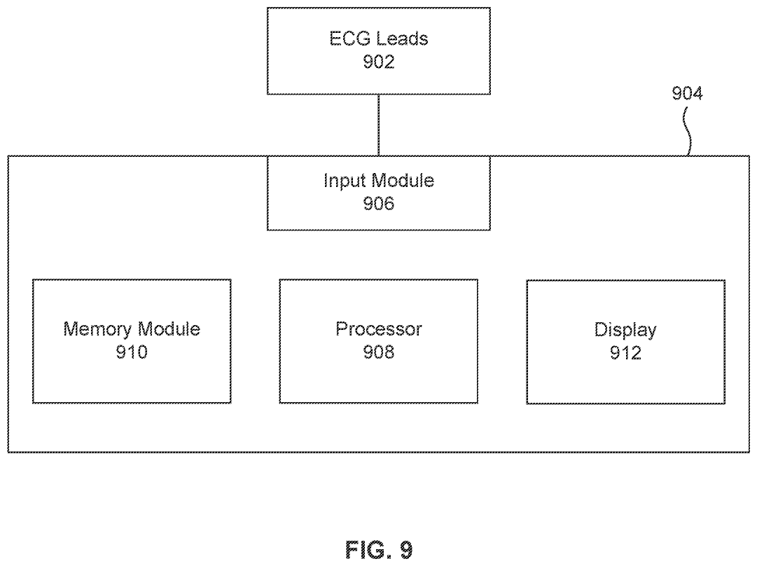

FIG. 9 illustrates an example ECG system 900 configured to perform the embodied multi-lead residuum procedure. ECG system 900 may be used at a hospital or may be a portable device for use wherever the patient may be. In another example, ECG system 900 may be an implantable biomedical device with leads implanted in various locations around the body of a patient. ECG system 900 may be part of or may be coupled with other implantable biomedical devices such as a cardiac pacemaker, an implantable cardioverter-defibrillator (ICD) or a cardiac resynchronization therapy (CRT) device. In the case of ICD or CRT devices, analysis of the residuum signal will be analyzed after inverse filtering of the ECG signal to offset device-specific ECG filters and reconstruct the device output.

ECG system 900 includes leads 902 and a main unit 904. Leads 902 may comprise any number and type of electrical lead. For example, leads 902 may comprise ten leads to be used with a standard 12-lead ECG. Leads 902 may be similar to leads 104a-j as illustrated in FIG. 1 and described previously. In another example, leads 902 may comprise implanted electrical leads, such as insulated wires placed throughout the body.

Main unit 904 may include an input module 906, a processor 908, a memory module 910 and a display 912. Input module 906 includes suitable circuitry and hardware to receive the signals from leads 902. As such, input module 906 may include components such as, for example, analog-to-digital converters, de-serializers, filters, and amplifiers. These various components may be implemented to condition the received signals to a more suitable form for further signal processing to be performed by processor 908.

It should be understood that in the case of the embodiment where ECG system 900 is an implantable biomedical device, display 912 may be replaced with a transceiver module configured to send and receive signals such as radio frequency (RF), optical, inductively coupled, or magnetic signals. In one example, these signals may be received by an external display for providing visual data related to measurements performed by ECG system 900 and analysis performed after inverse filtering of the received signal to reconstruct the signal following filtering by the device.

Processor 908 may include one or more hardware microprocessor units. In an embodiment, processor 908 is configured to perform signal processing procedures on the signals received via input module 906. For example, processor 908 may perform the multi-lead residuum procedure as previously described for aiding in the prediction of heart arrhythmias. Processor 908 may also comprise a field-programmable gate array (FPGA) that includes configurable logic. The configurable logic may be programmed to perform the multi-lead residuum procedure using configuration code stored in memory module 910. Likewise, processor 908 may be programmed via instructions stored in memory module 910.

Memory module 910 may include any type of memory including random access memory (RAM), read-only memory (ROM), electrically-erasable programmable read-only memory (EEPROM), FLASH memory, etc. Furthermore, memory module 910 may include both volatile and non-volatile memory. For example, memory module 910 may contain a set of coded instructions in non-volatile memory for programming processor 908. The calculated baseline signal may also be stored in either the volatile or non-volatile memory depending on how long it is intended to be maintained. Memory module 910 may also be used to save data related to the calculated TWH or RWH, including trend data for each.

In an embodiment, main unit 904 includes display 912 for providing a visual representation of the received signals from leads 902. Display 912 may utilize any of a number of different display technologies such as, for example, liquid crystal display (LCD), light emitting diode (LED), plasma or cathode ray tube (CRT). An ECG signal from each of leads 902 may be displayed simultaneously on display 912. In another example, a user may select which ECG signals to display via a user interface associated with main unit 904. Display 912 may also be used to show data trends over time, such as displaying trends of the calculated RWH and TWH.

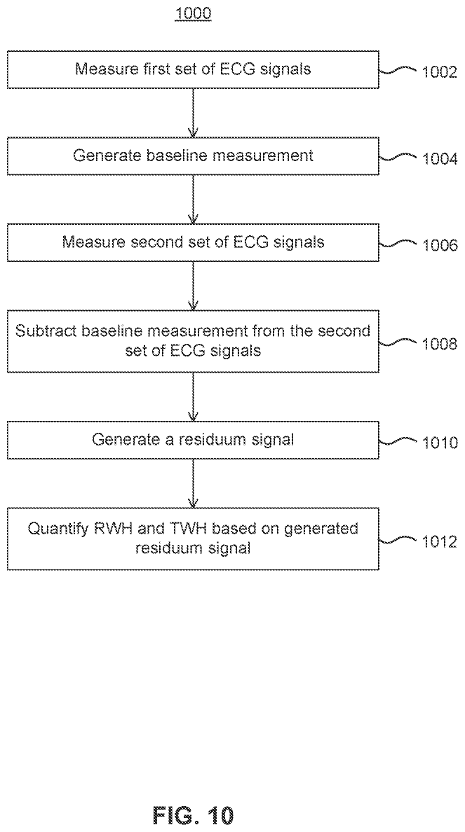

FIG. 10 illustrates a flowchart depicting a method 1000 for predicting heart arrhythmias based on RWH and TWH, according to an embodiment. Method 1000 may be performed by the various components of ECG system 900. It is to be appreciated that method 1000 may not include all operations shown or perform the operations in the order shown.

Method 1000 begins at step 1002 where a first set of ECG signals is monitored from a patient. The signals may be monitored via leads such as those illustrated in FIG. 1, or via implantable leads.

At step 1004, a baseline measurement associated with the morphology of the measured first set of ECG signals is generated. The baseline measurement may be generated by computing a median-beat sequence as described previously. The baseline measurement may be calculated, for example, over a period of 5 to 10 minutes in order to achieve a stable baseline signal. In an embodiment, a baseline measurement is generated for each lead of the standard 12-lead ECG.

At step 1006, a second set of ECG signals is monitored from the patient. The second set of signals may be monitored directly after monitoring the first set of signals or at any time after monitoring the first set of signals.

At step 1008, the baseline measurement is subtracted from the second set of monitored ECG signals, according to an embodiment. Each baseline measurement beat may be lined up either temporally or spatially with the various beats from each collected ECG signal for each lead in order to subtract the morphologies from one another. In another embodiment, the second set of monitored ECG signals may be divided by the baseline measurement on a point-by-point basis. Step 1008 may be performed independently for each lead of the standard 12-lead ECG using the baseline signal generated for each associated lead.

At step 1010, a residuum signal is generated for each lead based on the operation performed in step 1008 (e.g., subtraction or division according to the example embodiments described above). The residuum signal may be used to identify microvolt-level signal changes in particular segments of the ECG signal that would be otherwise difficult to detect.

At step 1012, RWH and TWH are quantified based on the generated residuum signals. In an embodiment, the residuum signals are calculated from each lead and the second central moment is derived for determining RWH and TWH.

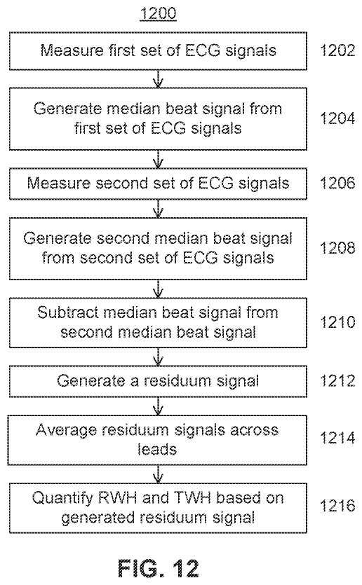

FIG. 12 illustrates a flowchart depicting another method 1200 for predicting heart arrhythmias based on RWH and TWH, according to an embodiment. Method 1200 may be performed by the various components of ECG system 900. It is to be appreciated that method 1200 may not include all operations shown or perform the operations in the order shown. Method 1200 enables high-throughput analysis of patient ECGs for determining arrhythmia risk.

Method 1200 begins at step 1202 where a first set of ECG signals is monitored from a patient. The signals may be monitored, for example, via external leads such as those illustrated in FIG. 1 or via implantable leads in various configurations or combinations.

At step 1204, a baseline measurement associated with the morphology of the measured first set of ECG signals is generated. The baseline measurement may be generated by computing a median-beat sequence as described previously. The baseline measurement may be calculated, for example, over a period of 5 to 10 minutes in order to achieve a stable baseline median beat signal. In an embodiment, a baseline measurement is generated for each lead of the standard 12-lead ECG. The baseline measurement may include only a single median beat.

At step 1206, a second set of ECG signals is monitored from the patient. The second set of signals may be monitored directly after monitoring the first set of signals or at any time after monitoring the first set of signals.

At step 1208, a median beat associated with the morphology of each ECG signal of the second set of ECG signals (i.e., a second median beat for each second ECG signal) is generated. A different second median beat may be calculated for each lead used to collect the second set of ECG signals. The median beat may be calculated, for example, over a period of 10 seconds.

At step 1210, the baseline median beat for each lead is subtracted from the second median beat for each lead of the second set of ECG signals. Each baseline median beat may be lined up either temporally or spatially with each second median beat of the second set of ECG signals in order to subtract the morphologies from one another.

At step 1212, a residuum signal is generated for each lead based on the subtraction performed in step 1210. The residuum signal may be used to identify microvolt-level signal changes in particular segments of the ECG signal that would be otherwise difficult to detect.

At step 1214, the residuum signals are averaged across each of the leads to generate an average residuum signal.

At step 1216, RWH and TWH are quantified based on the generated residuum signals and the average residuum signal. In an embodiment, the residuum signals are calculated from each lead and the second central moment is derived for determining RWH and TWH.

Either of methods 1000 or 1200 may be realized as a computer program product stored on a computer readable media. The computer program product includes a set of instructions that, when executed by a computing device, such as processor 908, perform the series of steps illustrated as part of either method 1000 or method 1200. Additionally, the instructions may include operations for measuring T-wave alternans (TWA) and determining trends of peak TWA, TWH and RWH values. The trends may be used to predict the onset of various heart arrhythmias, such as ventricular tachycardia.

### Using TWH to Determine Presence of Non-Flow Limiting Coronary Artery Stenosis and Diffuse Atherosclerosis or Microvascular Disease

Medical records from patients enrolled in RAND-CFR study at Brigham & Women's Hospital (Boston, Mass.) were analyzed. The study and medical records review were approved by the Partners Healthcare Institutional Review Board. Nondiabetic patients who performed a treadmill ETT followed by coronary angiography that confirmed ≤50% coronary artery stenosis from 2010 to 2016 at the Beth Israel Deaconess Medical Center (Boston, Mass.) constituted the control arm. The medical records analysis was performed under a protocol approved by the Beth Israel Deaconess Medical Center's Institutional Review Board.

The cases consisted of all 20 subjects enrolled in the RAND-CFR clinical trial whose ECGs during the no-drug phase at both rest and exercise could be analyzed. Eligible patients had diabetes, stable angina and/or exertional dyspnea during supine bicycle stress testing with exercise tolerance of ≥3 metabolic equivalents, and perfusion sum stress score<4 assessed by initial positron emission tomography (PET). Patients were excluded if they had obstructive CAD (≥50% stenosis on invasive coronary angiography within the past year or on study protocol-mandated screening coronary CT angiography), Seattle Angina Questionnaire score<100 and/or Rose Dyspnea Scale=0, history of cardiomyopathy, moderate or severe valvular heart disease, uncontrolled hypertension, kidney disease, or a contraindication for ranolazine.

The control group consisted of all nine nondiabetic subjects screened from medical records who had completed a symptom-limited treadmill ETT for suspected CAD over the past 5 years and in whom ≤50% coronary artery stenosis was subsequently confirmed by coronary angiography within 6 months after the ETT. Patients were excluded from the control group if they had any flow-limiting lesions (FFR<0.80), >50% stenosis of 2 or 3 vessels, >70% stenosis of any coronary artery or >50% of the left main coronary artery, moderate-to-severe valvular disease, chronic kidney disease, history of myocardial infarction, or cardiomyopathy.

Cases performed a symptom-limited supine bicycle stress test on a standard ramp protocol. Recordings of 12-lead ECGs (25 mm/s, 10 mV/mm) were printed for 12 s during rest, peak exercise, immediately postexercise, and at 1 min of recovery. Blood pressure and heart rate were measured at each stage.

Patients from the control group performed a symptom-limited treadmill ETT on a standard Bruce protocol with 3-min interval recordings of 12-lead ECGs, blood pressure, and heart rate. To exclude recordings with a high degree of motion artifact, stress ECGs (50 mm/s, 20 mV/mm) were analyzed for 6 s during the first 10 s of recovery while the subjects' heart rates remained high.

Standard 12-lead analog ECGs for all cases (25 mm/s, 10 mV/mm) and controls (50 mm/s, 20 mV/mm) were scanned with a high-resolution scanner. Patients without a complete set of left ECG leads V₄, V₅, or V₆, which were used for TWH calculation, or whose tracings had significant noise artifact or baseline wander were excluded. Image processing software, “ECGScan” (AMPS-LLC, New York, N.Y.), was then used to extrapolate the ECG waveforms using an active contour modeling technique.

The ECG measurements from left leads V₄, V₅, and V₆ were plotted and superimposed. It should be understood that right ECG leads V₁, V₂, and V3 could also be used to calculate TWH associated with the right portion of the heart (while the TWH measurement from leads V₄, V₅, and V₆ is associated with the left portion of the heart.) Second central moment analysis was performed on the JT interval to calculate TWH for every beat. The maximum splay in microvolts about the mean waveform from the J-point to end of the T wave (JT interval) during rest and exercise was reported for each patient. Since TWH is measured over the entire JT waveform, it does not depend on the specific T-wave endpoint as do time-dependent indices of dispersion of repolarization such as Tₚₑₐₖ−T_{end} or QT_{c} intervals.

Tₚₑₐₖ−T_{end} and QT intervals were measured primarily on lead V₅. If the amplitude of the T waves was <1.5 mm, lead V₅ was excluded from the analysis, and measurements were performed on lead V₄. If lead V₄ was also not suitable, lead V₆ was used instead. Tₚₑₐₖ−T_{end} and QT intervals were measured for three consecutive beats and the mean was taken as the final value. Both Tₚₑₐₖ−T_{end} and QT intervals were corrected for heart rate using Bazett's formula and are reported as Tₚₑₐₖ−T_{endc} and QT_{c} intervals.

For both the patients and the control group, ST-segment measurements were taken directly from the physicians' final clinical report of the ETT. An ETT was considered positive in patients with ST segment depression of ≥1 mm horizontal or downsloping configuration in two contiguous leads and three consecutive beats at 80 ms after the J-point.

In RAND-CFR patients, coronary flow was measured using a PET-CT scanner (Discovery RX or STE LightSpeed 64, GE Healthcare, Milwaukee, Wis.) in response to exercise using 13N-ammonia as flow tracer. CFR was calculated as the ratio of left ventricular myocardial blood flow (ml/g/min) during stress compared to rest. To account for disparities in resting cardiac workload, the CFR value at rest was corrected by the rate-pressure product, where a CFR<2.0 is considered to be hemodynamically significant. FFR was measured in control patients during cardiac catheterization using pressure wire assessment of identified narrowed segments in coronary arteries, using FFR>0.80 to determine absence of inducible ischemia.

All continuous variables were normally distributed according to Shapiro-Wilk normality test. Statistical differences between cases and controls were calculated using 2-tailed unpaired Student's t-tests. TWH was evaluated during rest and exercise for every patient, and the results within each group were compared using paired Student's t-tests. Bonferroni correction was performed for multiple comparisons within or between groups. Categorical variables were analyzed using Fisher's exact test. Two-tailed p-values<0.05 were considered significant.

Subjects from the RAND-CFR study all had diabetes (p<0.001) and were older (p=0.03) than the control group. Although the RAND-CFR patients had a higher prevalence of hypertension (p=0.03) than the control subjects, treatment achieved systolic and diastolic blood pressures within guidelines. Hyperlipidemia was present in 95% of cases with corresponding statin therapy as indicated in Table 1 below. Although resting heart rates were higher (p=0.02) in the RAND-CFR group, rate pressure products at rest (p=0.39) and during ETT (p=0.30) were similar to controls. The incidence of ETT-based ST-segment depression did not differ (p=0.11) between the groups.

TABLE 1
Patient characteristics

| | Controls | | Cases | | |
|---|---|---|---|---|---|
| | (N = 9) | | (N = 20) | | p |
| Age (years) | 57 ± 3 | | 64 ± 1 | | 0.06 |
| Sex (M/F) | 4/5 | | 9/11 | | 1 |
| BMI | 31 ± 2 | | 34 ± 2 | | 0.21 |
| Cardiovascular risk factors | | | | | |
| Diabetes (n, %) | 0 | | 20 | (100%) | <0.001* |
| Hypertension (n, %) | 3 | (33%) | 16 | (80%) | 1 |
| Smoking (n, %) | 4 | (44%) | 11 | (55%) | 1 |
| Hyperlipidemia (n, %) | 5 | (56%) | 19 | (95%) | 0.6 |
| Drug Therapy | | | | | |
| Beta-blockers (n, %) | 4 | (44%) | 13 | (65%) | 1 |
| Calcium antagonists (n, %) | 0 | | 5 | (25%) | 1 |
| ACEI and/or ARB (n, %) | 1 | (9%) | 16 | (80%) | 0.04* |
| Statins (n, %) | 4 | (44%) | 19 | (95%) | 0.4 |
| Antiaggregants (n, %) | 5 | (56%) | 15 | (75%) | 1 |
| Nitrates (n, %) | 4 | (44%) | 4 | (20%) | 1 |
| Hemodynamics at Rest | | | | | |
| SBP (mmHg) | 140 ± 7 | | 127 ± 3 | | 0.3 |
| DBP (mmHg) | 84 ± 3 | | 64 ± 2 | | <0.001* |
| HR (beats/min) | 60 ± 3 | | 72 ± 3 | | 0.09 |
| RPP (beats/min · mmHg) | 8440 ± 621 | | 9155 ± 467 | | 1 |
| Peak Exercise | | | | | |
| SBP (mmHg) | 178 ± 8 | | 175 ± 4 | | 1 |
| DBP (mmHg) | 84 ± 3 | | 79 ± 3 | | 1 |
| HR (beats/min) | 138 ± 6 | | 128 ± 4 | | 1 |
| RPP (beats/min · mmHg) | 24545 ± 1387 | | 22784 ± 938 | | 1 |
| ETT ST-segment | | | | | |
| depression ≥ 1 mm | | | | | |

| | | | |
|---|---|---|---|
| Positive | 5 | 4┐ | 0.11 |
| Negative | 4 | 14┘ | |

Key: BMI: body mass index, ACEI: angiotensin-converting enzyme inhibitors, ARB: angiotensin-II receptor blockers. SBP: systolic blood pressure, DBP: diastolic blood pressure, HR: heart rate, RPP: rate pressure product, ETT: exercise tolerance test.
*p < 0.05.

Corrected CFR values in the RAND-CFR group ranged from 0.94 to 2.57 (median=1.44), and 80% of the patients had CFR<2. The mean stenosis in the control group was 41±6% and 100% of the subjects had FFR≥0.80 (0.93±0.02).

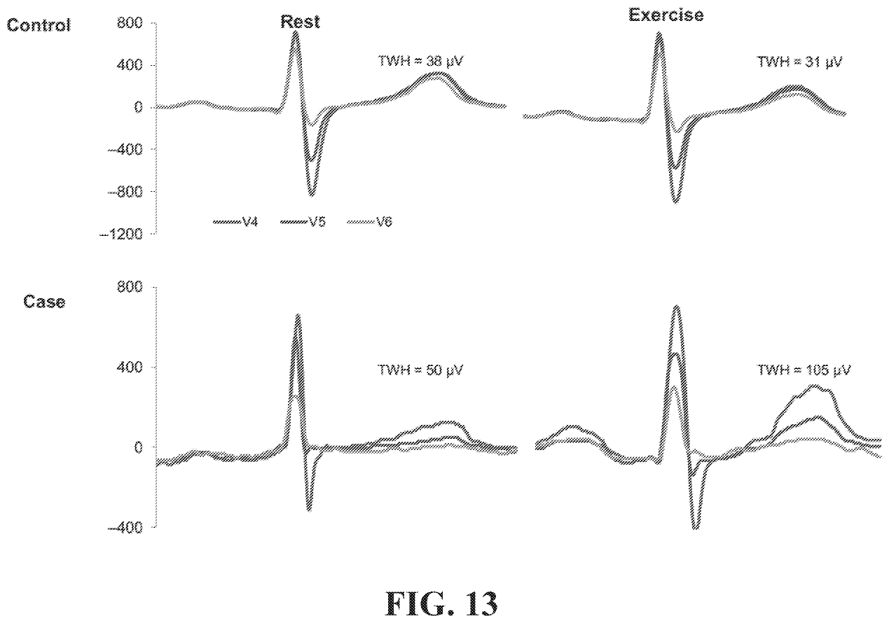

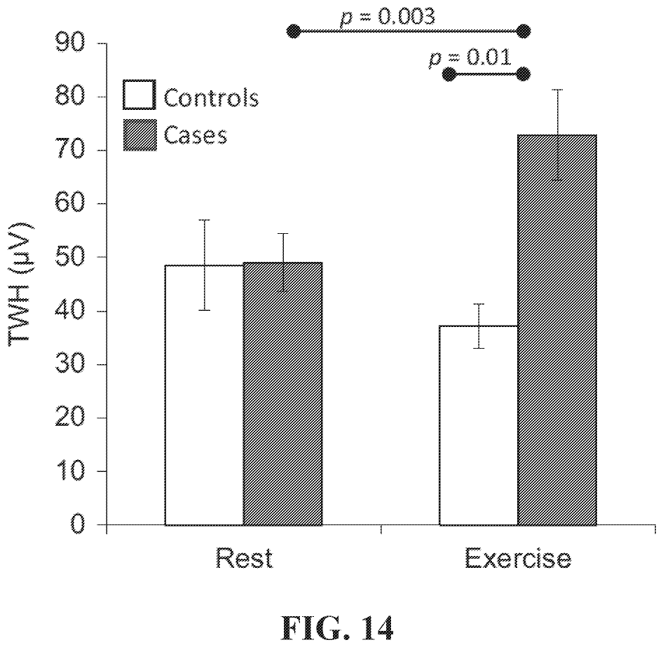

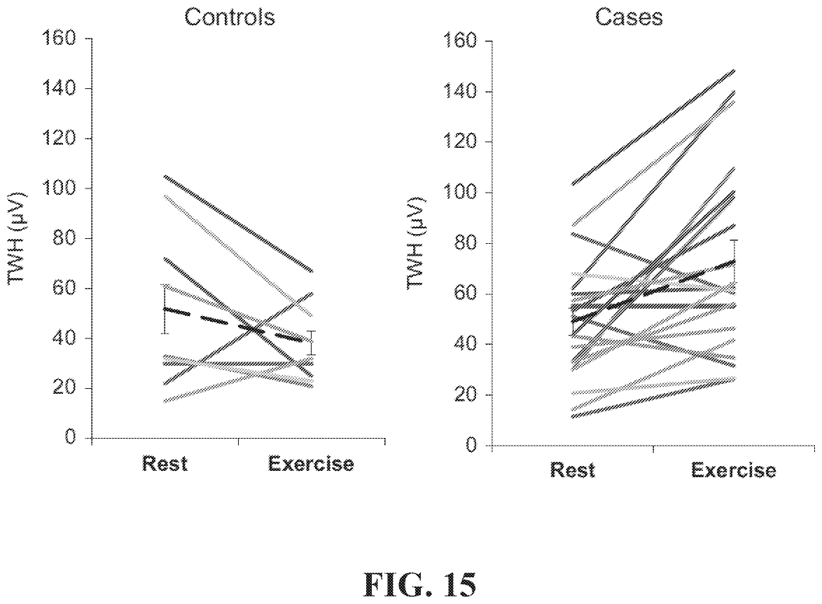

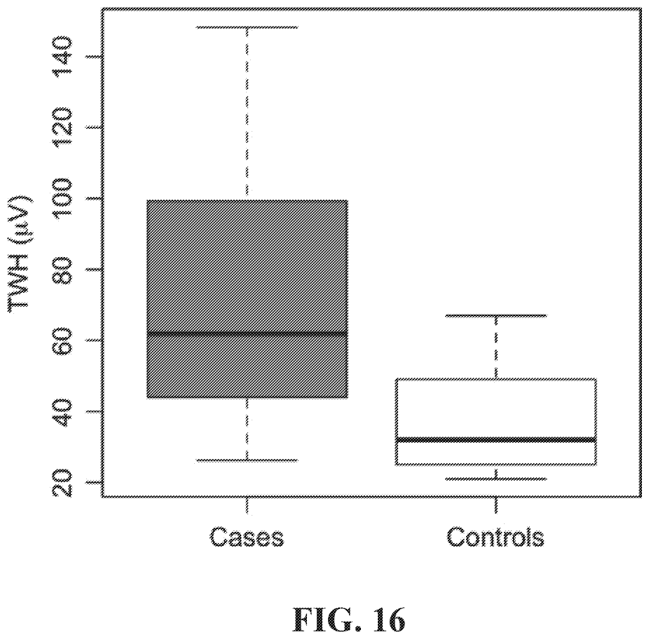

Representative digitized ECG tracings for a control subject and a RAND-CFR patient are provided in FIG. 13. At rest, TWH did not differ between RAND-CFR patients and the control group (controls: 52±11 μV; cases: 49±5 μV; p=0.80) (FIG. 14). With exercise, TWH increased by 49% to 73±8 μV (p=0.003) in RAND-CFR patients, while control subjects showed no change in TWH (to 38±5 μV, p=0.19) (FIGS. 14 and 15). More than 85% of RAND-CFR patients registered exercise TWH values above the median of controls (32 μV) and 70% were above the 3rd quartile of controls (49 μV) (FIG. 16). Exercise TWH values among RAND-CFR patients compared to control subjects were found not to be affected by use of ACEI or ARB (p=0.46) or by diastolic blood pressure (linear regression analysis, p=0.25). TWH values at rest were not different before randomization and after placebo (no drug: 49±5 μV, placebo: 46±5 μV; p=0.15), confirming reproducibility of the measurement. No statistically significant correlation between CFR and TWH was found during rest or exercise.

Tₚₑₐₖ−T_{end} values at rest did not differ between controls and the RAND-CFR group (controls: 80.7±6.2 ms; cases: 65.6±1.6 ms; p=0.06). Tₚₑₐₖ−T_{end} did not change with exercise in either group (controls: 82.9±16.8 ms, p=0.7; cases: 70.3±2.6 ms, p=0.18) and the final exercise Tₚₑₐₖ−T_{end} was not different between the two groups (p=0.16). When corrected for heart rate, Tₚₑₐₖ−T_{endc} was found to increase from rest to exercise in the RAND-CFR group but not in the control group (control: from 80±7 to 99±15 ms, p=0.07; cases: from 75.9±3 to 89±2.5 ms, p=0.008). Tₚₑₐₖ−T_{endc} did not differ between the two groups at either rest or exercise (p=0.6 and p=0.4 respectively).

QT_{c} intervals did not differ between the two groups at rest (controls: 403±15 ms; cases: 421±9 ms; p=0.28) and did not increase with exercise in the RAND-CFR group (to 438.3±6.3 ms, p=0.24) or in the control group (to 440±2 ms, p=0.06). During exercise, QT_{c} intervals also failed to discriminate cases from the control subjects (p=0.80).

Of the 20 cases in the RAND-CFR group, two did not have analyzable ST-segments due to left bundle branch block and were therefore excluded from this subanalysis. Exercise-induced ST-segment depression≥1 mm occurred in only four (22%) cases but in five (56%) controls. This finding suggests a high rate of false positive results in the controls, as FFR in all controls including these five individuals, four of whom were women, was >0.80.

The present study is the first to demonstrate a marked increase in exercise-induced TWH in symptomatic diabetic patients with nonflow-limiting coronary artery stenosis with diffuse atherosclerosis and/or microvascular dysfunction who were enrolled in the well-characterized RAND-CFR study. The increase in TWH was substantial (49%) from rest to ETT (49±5 to 73±8 μV, p=0.003). The level achieved approaches the 80-μV cutpoint associated with elevated risk for ventricular tachyarrhythmias and arrhythmic death. By comparison, nondiabetic control subjects with comparable degrees of nonflow-limiting coronary artery stenosis but with FFR>0.8 exhibited no significant ETT-induced change in TWH (rest: 49±8 μV; ETT: 37±4 μV, p=0.18) at an equivalent rate-pressure product. Contemporary indices of repolarization heterogeneity including Tₚₑₐₖ−T_{end} and QT_{c} failed to differentiate between cases and controls. The fact that most patients had abnormal CFR (with CFR<2 in 80%) as established by PET scanning while all control subjects had angiographically confirmed FFR>0.80, suggests a potential role of impaired supply-demand mismatch in the electrophysiologic basis for TWH.

Selection of the main ECG marker employed in the present study, TWH, was guided by results in preclinical studies of acute myocardial ischemia with and without concurrent adrenergic stimulation in large animal models. Elevated levels of TWH were found to herald the onset of ventricular tachycardia and fibrillation. These findings are consistent with the observations that myocardial ischemia results in marked dispersion of action potential duration and nonuniformities of recovery of excitability, changes highly conductive to malignant cardiac arrhythmias. Recently, the utility of TWH has been tested clinically and has been shown capable of detecting arrhythmia risk in diverse conditions including decompensated heart failure, ischemic and nonischemic cardiomyopathies, and in a population survey. It is germane that in both the experimental and clinical settings, TWH provides early signs of myocardial electrophysiologic dysfunction preceding the development of TWA and arrhythmias.

The precise factors that may have contributed to the increase in TWH during exercise in the present cohort of symptomatic patients with diabetes are likely multifold, given the complexity of this disease condition. Among the most prominent are the presence of coronary microvascular dysfunction, diffuse atherosclerosis, changes in myocardial structure including myofibrillar disarray induced by recurrent ischemic episodes, and altered autonomic function. That structural abnormalities are present in diabetic patients is supported by findings that QT dispersion, an indicator of heterogeneity of repolarization, is elevated during rest. Specifically, diabetes is associated with a decrease in high frequency (HF) heart rate variability (HRV) and increase in low/high (LF/HF) frequency HRV ratio. Elucidation of the relative contributions of each of these putative pathophysiologic mechanisms to exercise-induced increases in TWH as observed in the present study will require systematic investigation.

In terms of ST-segment changes during exercise, there was a relatively high incidence of false positives, as five (56%) controls had ST-segment depression>1 mm, despite FFR>0.8, indicating the absence of inducible ischemia. Of the five control subjects with depressed ST segments, four were female, consistent with high rate of false-positive results in women. In these four female control subjects, TWH was unchanged during exercise, consistent with FFR in the normal range. This pattern of response is at variance among cases with impaired CFR, in whom there was a marked increase in TWH in response to exercise (FIGS. 14 and 15).

Other contemporary risk markers, including ST-segment and Tₚₑₐₖ−T_{end} and QT intervals did not change significantly in response to exercise either in the control or RAND-CFR groups.

The main limitation of the current study is the relatively small size of the groups. However, the patients were carefully characterized by coronary angiography in the control subjects and by PET scan in cases. Two different ETT protocols were performed, namely, treadmill testing by control subjects and supine ergometry by the cases. However, the physiologic challenge during exercise was comparable given that the rate-pressure products achieved did not differ (Table 1).

Thus, TWH disclosed latent repolarization abnormalities during ETT in symptomatic diabetic patients with diffuse atherosclerosis and/or microvascular dysfunction that are not present in nondiabetic control subjects during rest or exercise despite similar levels of non-flow limiting coronary artery stenosis. The capacity of second central moment analysis to quantify TWH during ETT is an inherent advantage over other contemporary heterogeneity markers of sudden cardiac death risk, which are limited to use in patients in the resting state. The new technique developed in the current study, which enables analysis of archived ECGs, permits mining of extensive databases for retrospective studies and hypothesis testing.

### Using TWH to Determine Flow-Limiting Coronary Artery Stenosis

A study was performed to evaluate the capacity of TWH to detect the presence of large epicardial coronary vessel disease during either exercise tolerance testing (ETT) or pharmacologic stress testing. It is well-established that in patients with CAD, the supply-demand relationships would be impaired during either the workload stress of ETT or the coronary steal effect associated with dipyridamole challenge. The patient cohort consisted of individuals referred for these standard tests who underwent coronary angiography during cardiac catheterization within 0 to 5 days following the stress test. The results of this application of TWH evaluation were compared to those obtained based on standard ST-segment analysis.

Medical records and electrocardiograms from all 96 patients who performed either a treadmill ETT or dipyridamole pharmacological stress testing followed within 0 to 5 days by coronary angiography in 2016 at Beth Israel Deaconess Medical Center (Boston, Mass.) were analyzed. The medical records study was performed under a protocol approved by the Beth Israel Deaconess Medical Center's Institutional Review Board.

Cases consisted of 62 subjects whose coronary angiography revealed ≥50% stenosis of the left main coronary artery or ≥70% stenosis of an epicardial coronary vessel≥2 mm in diameter. Controls consisted of patients (N=34) who did not meet these criteria. Patients were excluded from the control group if they had moderate-to-severe valvular disease, chronic kidney disease, history of myocardial infarction, or cardiomyopathy.

ETT patients performed a symptom-limited treadmill ETT on CASE machines (GE Medical Systems Information Technologies, Inc., Milwaukee, Wis.) on a standard Bruce protocol with 3-min interval recordings of 12-lead ECGs, blood pressure, and heart rate. Pharmacological stress testing patients performed a symptom-limited intravenous (IV) dipyridamole stress test followed by cardiac MRI.

ETT ECGs (50 mm/s, 10 mV/mm) were analyzed in the 15 seconds preceding the beginning of treadmill exercise, 15 seconds after stopping exercise, and 15 seconds after a 5-minute interval following cessation of exercise. Pharmacologic stress ECGs (50 mm/s, 10 mV/mm) were analyzed in the 15 seconds preceding the beginning of dipyridamole infusion, 15 seconds after ending infusion, and 15 seconds after a 5-minute interval following cessation of infusion. Digital files were obtained from the GE machines and exported to the XML file format. Second central moment analysis was performed on the JT interval in precordial leads by a single person blinded to the clinical data to calculate TWH for every beat. The maximum splay in microvolts about the mean waveform from the J point to end of the T wave (JT interval) for TWH during rest and peak exercise was reported for each patient. Since TWH is measured over the entire JT waveform, it does not depend on the specific T-wave endpoint as do time-dependent indices of dispersion of repolarization such as Tₚₑₐₖ−T_{end} or QT_{c} intervals.

ST-segment measurements were taken directly from the stress test final clinical report. A stress test was considered positive in patients with ST segment depression of ≥1 mm horizontal or downsloping configuration in two contiguous leads and three consecutive beats at 80 ms after the J-point. Angiographic results were interpreted by a single investigator who did not have access to TWH results.

Statistical analyses were performed using GraphPad Prism 7 (GraphPad Software, Inc., La Jolla, Calif.). Data are reported as means±standard error of the mean (SEM). All continuous variables were normally distributed according to Shapiro-Wilk normality test. Statistical differences between cases and controls were calculated using 2-tailed unpaired Student's t-test or Welch's t-test, chosen accordingly through the application of the F-test for establishing the equality of variances. Results of TWH analyses within each group were compared using paired Student's t-tests. Bonferroni correction was performed for multiple comparisons within or between groups. Categorical variables were analyzed using Fisher's exact test. Receiver-Operator Characteristic (ROC) curves were plotted for ETT and pharmacological stress testing patients separately. Changes from rest to exercise in TWH values were used as plot variables. Two-tailed p-values<0.05 were considered significant.

The patient characteristics comparing controls with cases are provided in Table 2 below. There were no statistically significant differences among the two groups with respect to age, sex, hypertension, hyperlipidemia, congestive heart failure, or chronic kidney disease. The presence of diabetes in cases over controls was found to have borderline significance (p=0.050).

TABLE 2
Patient characteristics

| | Controls | | Cases | | |
|---|---|---|---|---|---|
| | (n = 34) | | (n = 62) | | P value |
| Age (years) | 61.3 | | 65.7 | | 0.58 |
| Male (n, %) | 19 | (56%) | 39 | (63%) | 0.52 |
| Female (n, %) | 15 | (44%) | 23 | (37%) | 0.52 |
| Diabetes (n, %) | 9 | (26%) | 30 | (48%) | 0.050 |
| Hypertension (n, %) | 21 | (62%) | 49 | (79%) | 0.081 |
| Hyperlipidemia (n, %) | 22 | (65%) | 51 | (82%) | 0.079 |
| Congestive heart failure (n, %) | 8 | (24%) | 13 | (21%) | 0.800 |
| Chronic kidney disease (n, %) | 4 | (12%) | 11 | (18%) | 0.563 |
| ETT (n, %) | 20 | (59%) | 42 | (68%) | 0.504 |
| ETT positive by ST-segment | 13 | (38%) | 28 | (45%) | 0.527 |
| Dipyridamole stress test (n, %) | 14 | (41%) | 20 | (32%) | 0.504 |
| Dipyridamole stress | 0 | (0%) | 4 | (6%) | 0.294 |
| test positive by ST- | | | | | |
| segment | | | | | |

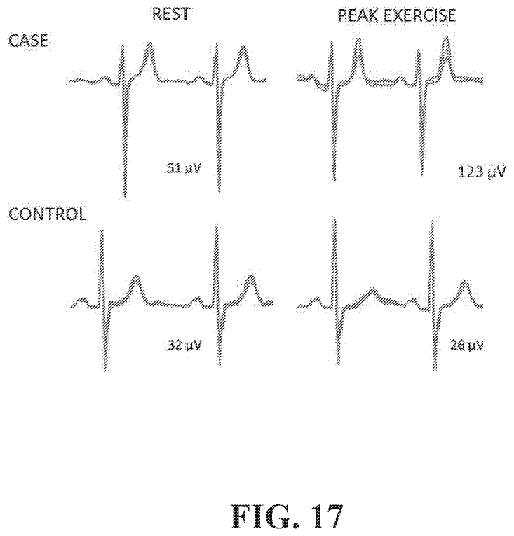

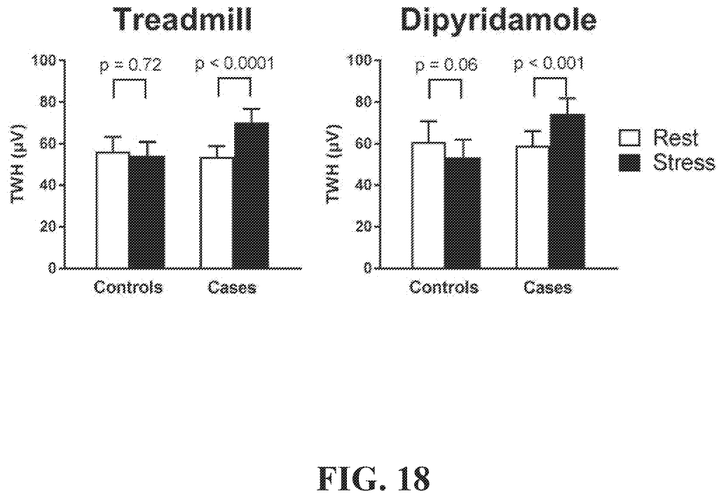

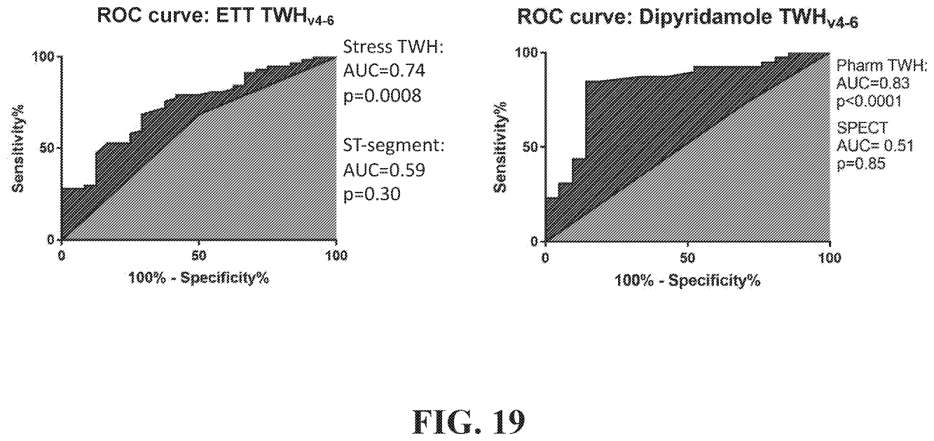

A representative example of TWH during rest and during ETT is provided in FIG. 17. At rest, TWH levels were similar for cases and controls as shown in FIG. 18. ETT and dipyridamole stress testing induced significant TWH increases (30%, p<0.0001; 26%, p<0.001, respectively) in cases. In controls, TWH did not change. Area under the receiver operator characteristic curve (AUC) for an increase in TWH as an indicator of flow-limiting coronary artery stenosis was 0.73 for ETT (p=0.003) and 0.88 for intravenous (IV) dipyridamole (p=0.0001) (FIG. 19). ST-segment changes with either ETT or dipyridamole did not discriminate cases from controls (AUC=0.56 for both tests, NS) as shown in FIG. 19.

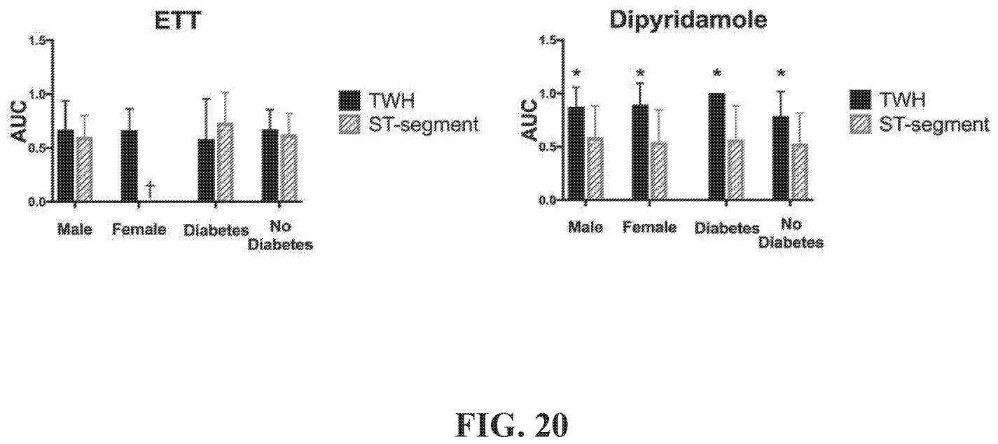

The superiority of TWH is also evident in the subgroup analysis, as AUCs were significantly greater for TWH than for ST-segment during dipyridamole stress testing in identifying the presence of epicardial coronary artery stenosis in men and women and in patients with and without diabetes, as shown in FIG. 20. TWH was more effective than ST-segment in detecting coronary artery stenosis in females during ETT than was ST-segment but differences between detection by TWH and ST-segment were not significant in males or in patients with or without diabetes during ETT (FIG. 20).

This is the first study to examine the potential utility of TWH to detect the presence of large epicardial coronary artery stenosis in the context of standard ETT and pharmacologic stress testing. The results of TWH analyses were compared to ST-segment measurements in relation to the “gold standard” of diagnostic catheterization described by Kern et al., “Physiological assessment of coronary artery disease in the cardiac catheterization laboratory: A scientific statement from the American Heart Association Committee on Diagnostic and Interventional Cardiac Catheterization, Council on Clinical Cardiology,” Circulation, 2006, 114:1321-1341, which was performed at 0 to 5 days after stress testing. In patients with significant CAD, defined as ≥50% stenosis of the left main coronary artery or ≥70% stenosis of an epicardial coronary artery≥2 mm in diameter, both ETT and dipyridamole tests resulted in a significant increase in TWH of 30% (p<0.0001) and 26% (p<0.001), respectively, as shown in FIG. 18. In the control group, which consisted of subjects who did not meet these CAD stenosis criteria, TWH was not altered in response to either stress test. ROC curves for the sensitivity/specificity relationship revealed AUC=0.73 (p<0.003) for ETT and AUC=0.88 (p<0.001) for dipyridamole testing. The performance of TWH during both tests was superior to ST-segment results, which yielded nonsignificant AUC's of 0.56 and 0.51, respectively.

A fundamental premise to be tested in this study is that during rest, ECG abnormalities associated with coronary artery stenosis may not be evident, but when the myocardial substrate is challenged by a stimulus such as exercise, which perturbs the supply-demand relationship and increases adrenergic drive, undisclosed epicardial coronary artery stenosis can be detected using the present novel approach. Consistent with this conceptual framework, at rest, TWH levels were not different in individuals with or without significant coronary artery stenosis. However, in response either to ETT or dipyridamole stress, TWH was substantially increased in subjects with obstructive coronary artery stenosis but not in control subjects. Receiver operator characteristic curve analysis of sensitivity/specificity relationships revealed that TWH with either stress test was superior to ST-segment (FIG. 19). It is noteworthy that the AUC for dipyridamole testing is superior to that of ETT (0.88 vs. 0.73, respectively).

Dipyridamole testing is based on inducing inhomogeneous perfusion through steal of coronary blood flow from diseased to normal zones. It is germane in this regard that in cases, dipyridamole induced increased TWH without a significant effect on ST-segment (as shown in Table 2). Thus, TWH is well suited for use with dipyridamole testing for the detection of flow-limiting coronary artery stenosis, as also shown by the results provided in FIG. 19.

The rationale for ETT is the imposition of a workload on the heart, which is achieved by increasing heart rate and systemic arterial pressure resulting in a challenge between supply and demand as well as an increase in cardiac sympathetic drive. The ETT test relies on patient motivation and physical capacity to exercise, as well as other factors that can impair chronotropic response such as bradycardia-inducing medications including beta-blockers and calcium channel antagonists.

The present study provides encouraging results that TWH can accurately determine the presence or absence of epicardial coronary stenosis in women, a particularly difficult group for stenosis detection. In fact, the AUC for the TWH response to dipyridamole was similar to that of men as shown in FIG. 20. AUCs for ST-segment detection of coronary artery stenosis during ETT for women were <0.10, consistent with the limitations of this parameter observed in other studies. One possibility for this finding is that while the postulated digitalis-like structure of estrogen may alter ST-segment pattern in individual leads, the splay in interlead morphology as evaluated by TWH may not be disrupted by this confounding influence, resulting in improved discrimination between the presence and absence of coronary artery stenosis. The presence or absence of diabetes did not alter the capacity to detect epicardial coronary artery stenosis in response to dipyridamole testing as shown in FIG. 20.

### Additional Measurements During ETT or Pharmacologic Stress Testing

It was further examined whether measurement of interlead T-wave heterogeneity (TWH) during exercise tolerance testing (ETT) or pharmacologic stress with dipyridamole during myocardial perfusion imaging (MPI) improves detection of stenoses in large epicardial coronary arteries. Despite widespread use of ETT and vasodilator-stress MPI for noninvasive detection of flow-limiting coronary artery disease, there is a continued need to improve diagnostic accuracy.

The methodology used was as follows. All 139 patients at the Beth Israel Deaconess Medical Center institution who underwent diagnostic coronary angiography within 0 to 5 days after exercise tolerance testing (ETT, N=81) or intravenous (IV) dipyridamole infusion (N=60) in 2016 were studied, including 2 patients with both tests. Cases (N=94) had angiographically significant stenosis (≥50% of left main or ≥70% of an epicardial coronary artery≥2 mm in diameter); controls (N=45) did not. TWH, i.e., interlead splay of T waves, was determined by second central moment analysis from precordial leads by investigators blinded to stress test ST-segment and angiographic results.

A summary of the results are as follows. At rest, TWH levels were similar for cases and controls. ETT and dipyridamole testing increased TWH significantly (by 68%, p<0.001, and 28%, p<0.001, respectively) in cases. In controls, TWH did not change. Areas under the receiver operating characteristic curve (AUC) for TWH increase for any flow-limiting coronary artery stenosis were 0.74 (p=0.0008) for ETT and 0.83 (p<0.0001) for dipyridamole. By contrast, ST-segment changes during ETT did not discriminate cases from controls (AUC=0.59, p=0.30); MPI during dipyridamole stress testing did not discriminate cases from controls (AUC=0.51, p=0.85).

The conclusion of the additional testing is as follows. TWH measurement is a novel method that improves the diagnostic accuracy of both ETT and pharmacologic stress testing with dipyridamole during MPI for detecting flow-limiting stenoses in large epicardial coronary arteries. ETT and pharmacological stress-induced T-wave heterogeneity (TWH), a measure of interlead splay of the waveforms, is superior to ST-segment changes in identifying patients with coronary artery stenosis warranting revascularization. Area under the receiver operating characteristic curve (AUC) for TWH for any flow-limiting coronary artery stenosis was 0.74 for ETT (p<0.001) and 0.83 for dipyridamole (p<0.0001). TWH measurement is a novel method that improves the diagnostic accuracy of both ETT and pharmacologic stress testing with dipyridamole during MPI for detecting flow-limiting stenoses in large epicardial coronary arteries and does not changes in routine stress protocols or specialized equipment or electrodes. The details of this additional testing are provided below.

Noninvasive detection of flow-limiting stenoses in large epicardial coronary arteries remains a daily challenge in contemporary cardiology. In addition to symptom evaluation, the main first-line diagnostic techniques for assessing coronary artery disease (CAD) are exercise tolerance testing (ETT) and pharmacological stress testing. Each test is conducted either independently or in conjunction with echocardiographic or nuclear myocardial perfusion imaging (MPI). The induction of ST-segment depression during ETT is the most widely employed objective sign of CAD-associated myocardial ischemia. However, during pharmacologic stress testing with MPI, ST-segment evaluation has proved unreliable and reversible perfusion defects are the main indicators of disease.

Notwithstanding an extensive experience, it is generally recognized that these tests in their current form yield excess numbers of both false positive and false negative results as compared with diagnostic coronary angiography. With regard to ETT, several other ECG parameters beyond ST-segment deviation have been evaluated to improve the diagnostic yield of stress testing, including ST/heart rate slope or index and ST/heart rate recovery loop. However, none has been shown to provide convincing diagnostic value in clinical practice. ETT-based detection of coronary artery stenosis using ST-segment measurement is particularly problematic in women due to the high false positive rate, which ranges from 25% to 50%.

Recently, a noninvasive technique termed “second central moment” analysis was developed to assess repolarization heterogeneity quantitatively by analyzing the interlead splay or variance of T-wave morphology simultaneously recorded in adjoining precordial leads with respect to the computed mean waveform. This parameter, designated “T-wave heterogeneity” (TWH), has undergone extensive testing in the experimental laboratory and was shown to be highly accurate in detecting arrhythmia susceptibility during acute myocardial ischemia. In the 5600-subject Health Survey 2000 study, which was designed to examine a cross-section of the entire Finnish population, heterogeneity was found to predict cardiac mortality and sudden cardiac death with odds ratios of 3.2 to 3.5. TWH was also found suitable to estimate risk for arrhythmia and mortality in patients with ischemic and nonischemic cardiomyopathy. However, TWH has not previously been evaluated for detection of clinically significant epicardial coronary artery stenoses with reference to diagnostic coronary angiography.

The main goal of the present study was to evaluate the capacity of TWH to detect the presence of large epicardial coronary artery stenosis warranting revascularization during either ETT or pharmacologic stress testing. It was hypothesized that the basis for its detection of CAD during ETT is impairment of the supply-demand relationship, which would be manifest as an increase in TWH in cases but not controls. In patients undergoing pharmacologic stress testing with dipyridamole, the attendant coronary steal effects of this vasodilator agent in patients with CAD would be expected to result in nonuniform changes in T-wave morphology in cases that can be quantified by TWH measurement. This hypothesis is based on the fact that during the early stage of even mild myocardial ischemia, changes such as peaked T-waves occur prior to alterations in ST-segment levels. It is germane in this regard that ischemia-induced changes in ATP-sensitive potassium channels (K_{ATP}), which are nonuniformly distributed, can markedly shorten action potentials resulting in heterogeneous changes in T-wave morphology across the heart.

Thus, it was postulated that the attendant physiological demands on diseased hearts imposed by ETT or pharmacologic stress testing with dipyridamole would result in increases in nonuniformities of spatial-temporal repolarization in cases but not controls, which can be quantified by TWH analysis, accordingly providing a method for coronary artery stenosis detection. The patient cohort consisted of individuals referred for these standard tests who underwent diagnostic coronary angiography within 0 to 5 days following the stress test. The results of this novel application of TWH evaluation were compared to those obtained based on standard ST-segment analysis and MPI.

Medical records and electrocardiograms from all 144 patients with interpretable ECGs who performed either a treadmill ETT or intravenous (IV) dipyridamole pharmacological stress testing followed within 0 to 5 days by coronary angiography in 2016 at Beth Israel Deaconess Medical Center (Boston, Mass.) were analyzed. Of the patients who fit the timeline, five control subjects exhibited markedly peaked T waves and correspondingly elevated TWH values at baseline. As outliers from the pattern of distribution, these control subjects were excluded from the overall analysis, leaving 139 patients in the study. Mean TWH levels did not differ after this exclusion. The medical records study was performed under a protocol approved by the Beth Israel Deaconess Medical Center's Institutional Review Board.

Cases consisted of 94 subjects whose coronary angiography revealed ≥50% stenosis of the left main coronary artery or ≥70% stenosis of an epicardial coronary vessel≥2 mm in diameter. Controls consisted of patients (N=45) who did not meet these criteria. Two patients who had both tests were included. Effects on TWH of right-sided coronary artery stenosis were analyzed in cases (N=59) with right-sided stenosis≥70% and controls (N=45), who were the same controls mentioned above.

### Exercise Tolerance Testing

ETT patients performed a symptom-limited ETT on CASE treadmills (GE Medical Systems Information Technologies, Inc., Milwaukee, Wis.) following a modified Bruce protocol with 3-min interval recordings of 12-lead ECGs, blood pressure, and heart rate. ETT ECGs were analyzed in the 15 seconds preceding the beginning of treadmill exercise (rest), 15 seconds after stopping exercise, and 15 seconds after a 5-minute interval following cessation of exercise.

Dipyridamole was infused for ˜4 min at a dose of 0.142 mg/kg/min IV. At 1 to 2 min after cessation of infusion, 31.6 mCi of Tc-99m sestamibi was injected IV. Gated SPECT stress images were obtained ˜30 min following tracer injection. Resting perfusion images were obtained on a subsequent day with Tc-99m sestamibi. Tracer was injected ˜45 min prior to obtaining the resting images. Results were interpreted with the 17-segment myocardial perfusion model. ECGs recorded during dipyridamole infusion were analyzed in the 15 seconds preceding the beginning of dipyridamole infusion, 15 seconds after ending infusion, and 15 seconds after a 5-minute interval following cessation of infusion. Digital files were obtained from the GE machines and exported using an XML file format. Computer software was written in the Python programming language to allow reading of the digital files and measurement of TWH.

Second central moment analysis was performed on the JT interval in precordial leads by a single author (A.S.) blinded to the clinical data to calculate TWH for every beat. TWH calculated in leads V₄, V₅ and V₆ is referred to as TWH_{V4-6}, while TWH calculated in leads V₁, V₂ and V₃ was designated TWH_{V1-3}. The maximum splay in microvolts about the mean waveform from the J point to end of the T wave (JT interval) for TWH during rest and after peak stress was reported for each patient. Since TWH is measured over the entire JT waveform, it does not depend on the specific T-wave endpoint as do time-dependent indices of dispersion of repolarization such as Tₚₑₐₖ−T_{end} or QT_{c} intervals.

ST-segment assessments were taken directly from the stress test final clinical report interpreted by exercise physiologists or cardiology fellows and overread by board certified cardiologists. A stress test was considered positive in patients with ST segment depression of ≥1 mm horizontal or downsloping configuration in two or more contiguous leads in three consecutive beats at 80 ms after the J-point. Angiographic results were interpreted by a single investigator who did not have access to TWH results and was blinded to the clinical stress test results.

### Statistics

Statistical analyses were performed using GraphPad Prism 7 (GraphPad Software, Inc., La Jolla, Calif.). Data are reported as means±standard error of the mean (SEM). All continuous variables were normally distributed according to Shapiro-Wilk normality test. Statistical differences between cases and controls were calculated using 2-tailed unpaired Student's t-test or Welch's t-test, chosen according to the application of the F-test for establishing the equality of variances. Results of TWH analyses within each group were compared using paired Student's t-tests. Bonferroni correction was performed for multiple comparisons within or between groups. Categorical variables were analyzed using Fisher's exact test. Receiver Operating Characteristic (ROC) curves were plotted separately for ETT and pharmacological stress test patients. Changes from rest to exercise in TWH values were used as plot variables. Two-tailed p-values<0.05 were considered significant.

TABLE 3
Patient characteristics

| | Controls | | Cases | | |
|---|---|---|---|---|---|
| | (n = 45) | | (n = 94) | | P value |
| Age (years) | 63.0 ± 2.0 | | 66.5 ± 1.1 | | 0.09 |
| Male (n, %) | 22 | (49%) | 62 | (66%) | 0.05 |
| Body mass index (kg/m²) | 31.4 ± 1.3 | | 30.9 ± 1.3 | | 0.81 |
| Diabetes mellitus (n, %) | 20 | (44%) | 43 | (46%) | 0.88 |
| Hypertension (n, %) | 29 | (64%) | 73 | (78%) | 0.09 |
| Hyperlipidemia/ | 28 | (62%) | 71 | (76%) | 0.10 |
| hypercholesterolemia (n, %) | | | | | |
| Congestive heart failure | 11 | (24%) | 20 | (21%) | 0.67 |
| (LVEF ≤ 35%) (n, %) | | | | | |
| Chronic kidney disease (n, %) | 5 | (11%) | 23 | (24%) | 0.06 |
| Atrial Fibrillation during | 3 | (7%) | 9 | (10%) | 0.57 |
| ETT or dipyridamole (n, %) | | | | | |
| Current smoking (n, %) | 1 | (2%) | 1 | (1%) | 0.59 |
| Drug therapy | | | | | |
| Beta-blockers (n, %) | 19 | (42%) | 51 | (54%) | 0.18 |
| Calcium antagonists (n, %) | 7 | (16%) | 20 | (21%) | 0.42 |
| ACE-I or ARB (n, %) | 16 | (36%) | 42 | (45%) | 0.30 |
| Statins (n, %) | 25 | (56%) | 64 | (68%) | 0.14 |
| Nitrates (n, %) | 4 | (9%) | 24 | (26%) | 0.02 |
| Exercise tolerance test (n, %) | 24 | (53%) | 57 | (59%) | |
| ETT-induced ST-segment | 12 | (27%) | 39 | (41%) | 0.11 |
| depression ≥ 1 mm | | | | | |

Key: BMI, body mass index; ETT, exercise tolerance test; ACEI, angiotensin-converting enzyme inhibitors; ARB, angiotensin-II receptor blockers.
*p < 0.05.

The patient characteristics comparing controls with cases are provided in Table 3. There were no statistically significant differences among the two groups with respect to age, diabetes mellitus, hypertension, hyperlipidemia, congestive heart failure, or chronic kidney disease. Cases differed from controls in terms of sex, more frequent prior diagnosis of coronary artery disease and prescription of nitrates. In cases, the systolic blood pressure, heart rate, and heart rate-blood pressure product during in ETT were lower in cases than controls (Table 4).

TABLE 4
Exercise tolerance test parameters

| | Controls | Cases | |
|---|---|---|---|
| | (n = 25) | (n = 55) | P value |
| Hemodynamics at rest in | | | |
| patients with ETT | | | |
| SBP (mmHg) | 128 ± 3.7 | 129 ± 2.7 | 0.86 |
| DBP (mmHg) | 77 ± 1.8 | 76 ± 1.3 | 0.55 |
| Heart rate (beats/min) | 69 ± 2.0 | 68 ± 1.4 | 0.51 |
| Rate-pressure product | 8925 ± 389.0 | 8802 ± 283.2 | 0.80 |
| (beats/min × mmHg) | | | |
| Hemodynamics at peak | | | |
| exercise | | | |
| SBP (mmHg) | 171 ± 6.3 | 156 ± 3.4 | 0.03 |
| DBP (mmHg) | 74 ± 2.1 | 70 ± 1.4 | 0.10 |
| Heart rate (beats/min) | 141 ± 4.5 | 125 ± 2.7 | 0.002 |
| Rate-pressure product | 24493 ± 1386.1 | 19718 ± 699.8 | 0.001 |
| (beats/min × mmHg) | | | |

Key: SBP, systolic blood pressure; DBP, diastolic blood pressure.

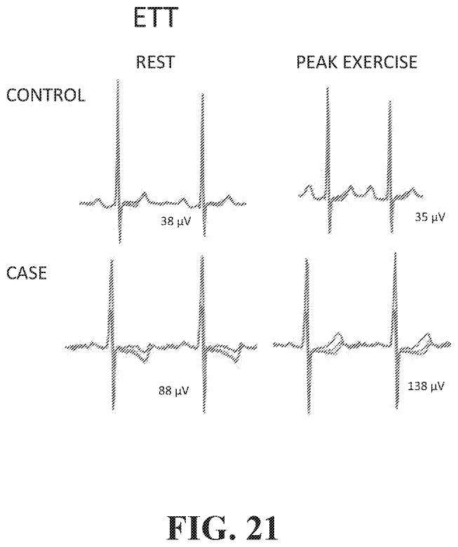

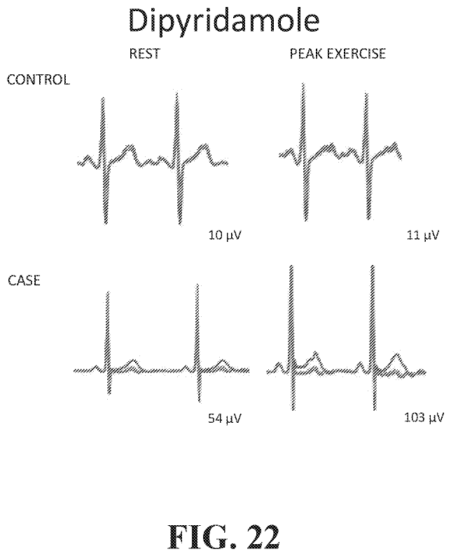

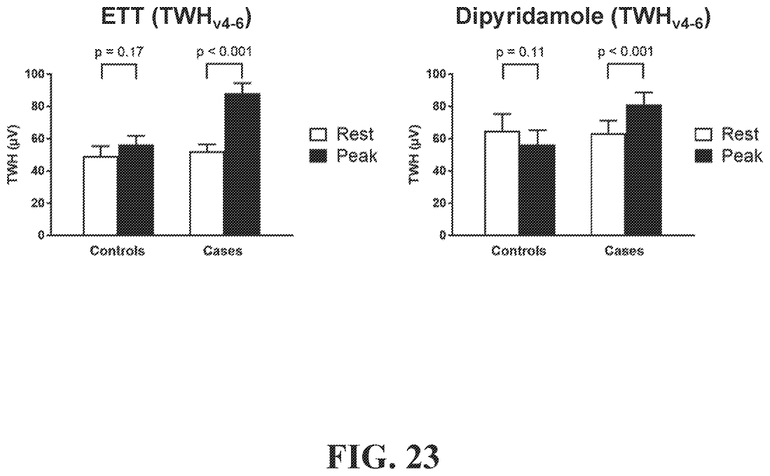

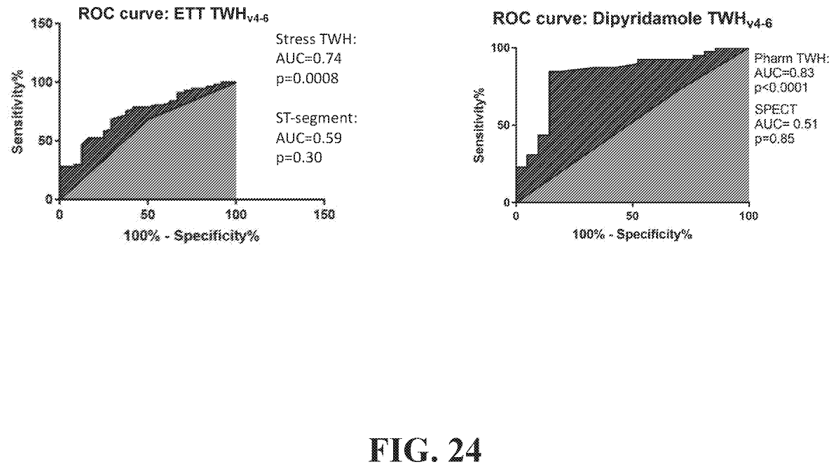

Representative examples of TWH during rest and during ETT (FIG. 21) or dipyridamole stress testing (FIG. 22) are provided. At rest, TWH_{V4-6} levels were similar for cases and controls (FIG. 23). ETT and dipyridamole stress testing induced significant TWH_{V4-6} increases (68%, p<0.0001; 28%, p<0.0001, respectively) in cases. In controls, TWH_{V4-6} did not change. Area under the receiver operator characteristic curve (AUC) for an increase in TWH_{V4-6} as an indicator of flow-limiting coronary artery stenosis was 0.74 for ETT (p<0.001) and 0.83 for dipyridamole (p<0.0001) (FIG. 24). ST-segment changes with either ETT or dipyridamole did not discriminate cases from controls (AUC=0.59 for ETT and AUC=0.52 for dipyridamole testing, NS) (FIG. 24).

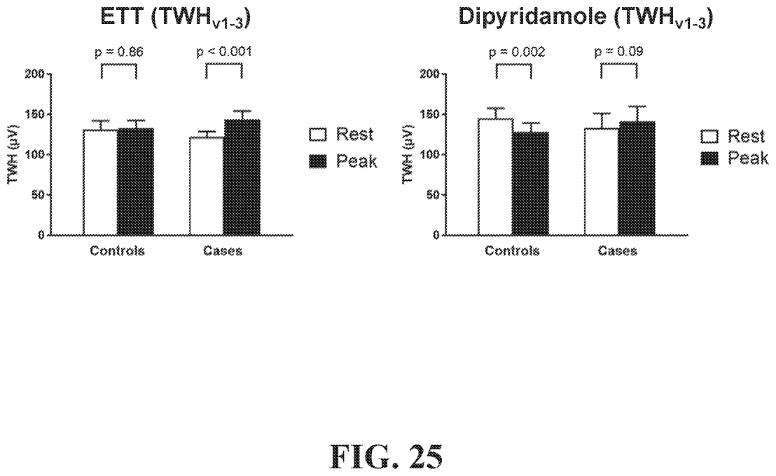

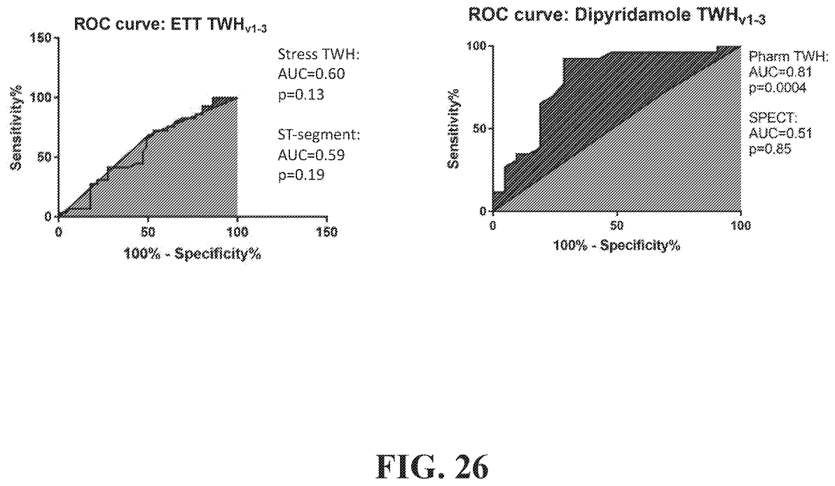

Capacity to detect right-sided coronary artery stenosis was analyzed in the right precordial leads (V₁, V₂, V₃). At rest, TWH_{V1-3} levels were similar for ETT cases and controls (FIG. 25). ETT testing induced significant TWH_{V1-3} increases (18%, p<0.001) in cases with right-sided disease. Dipyridamole testing induced significant TWH_{V1-3} decreases (12%, p=0.002) in controls but there was no significant difference in TWH_{V1-3} values in cases (p=0.09). Area under the receiver operator characteristic curve (AUC) for an increase in TWH_{V1-3} as an indicator of flow-limiting coronary artery stenosis was 0.60 for ETT (p=0.13) and 0.81 for dipyridamole testing (p<0.0001) (FIG. 26). ST-segment changes with either ETT or dipyridamole (light gray area only) did not discriminate cases from controls (AUC=0.59, p=0.19 for ETT, and AUC=0.52, p=0.79, for dipyridamole).

This is the first study to examine the potential utility of TWH to detect the presence of flow-limiting stenoses in large epicardial coronary arteries during standard ETT and pharmacologic stress testing with dipyridamole during MPI. The results of TWH analyses were compared to ST-segment measurements during ETT and to SPECT imaging results during MPI. TWH, ST-segment, and MPI findings were compared to results of diagnostic coronary angiography, which was performed at 0 to 5 days after stress testing. In patients with significant CAD, defined as ≥50% stenosis of the left main coronary artery or ≥70% stenosis of an epicardial coronary artery≥2 mm in diameter, both ETT and dipyridamole tests resulted in a significant increase in TWH_{V4-6} of 68% (p<0.0001) and 28% (p<0.0001), respectively (FIG. 23) compared with the resting baseline state. In the control group, which consisted of subjects who did not meet these stenosis criteria, TWH_{V4-6} was not altered in response to either stress test. ROC curves for the sensitivity/specificity relationship revealed AUC=0.74 (p<0.001) for ETT and AUC=0.83 (p<0.0001) for dipyridamole testing. The performance of TWH_{V4-6} during both tests was superior to ST-segment results, which yielded nonsignificant AUC's of 0.59 and 0.52, respectively.

### Prior Studies

Extensive experimental investigations support the concept that myocardial ischemia induces marked dispersion of action potential duration and nonuniformities of recovery of excitability, establishing an electrophysiologic milieu that is conducive to life-threatening cardiac arrhythmias. These fundamental observations prompted clinical investigations to determine whether repolarization heterogeneity could be utilized to assess risk for lethal arrhythmias.

Several investigators explored the QT dispersion method introduced by Higham and Campbell in the 1990's (1994). The concept is relatively straightforward, involving determining the greatest difference in the QT interval across the standard 12-lead recordings. The results proved somewhat encouraging, with the demonstration that ETT elicits significant degrees of QT dispersion in patients with compared with without ischemic heart disease. In post-menopausal women, a challenging population for myocardial ischemia detection, the occurrence of high levels of QT dispersion significantly improved the sensitivity and specificity of ETT testing over ST-segment changes alone for the presence of CAD. In recent years, however, QT dispersion has fallen out of favor due to the recognition of significant conceptual and methodological limitations. These relate in part to difficulties in accurately determining the end of the T wave and doubt concerning whether QT interval differences across the 12-lead electrode set accurately represents the electrophysiologic property of dispersion.

To circumvent these methodologic difficulties and conceptual limitations, an approach in which the entire waveform of adjoining leads is assessed was developed, termed T-wave heterogeneity or “TWH.” The approach involves quantification of the splay in interlead morphology using second central moment analysis. The capacity of TWH to detect the presence and arrhythmogenic consequences of myocardial ischemia in response to both total and non-occlusive stenoses of large epicardial coronary vessels including the left anterior descending and left circumflex coronary arteries has been extensively tested in large animal models. In the clinical setting, TWH has been evaluated under diverse ischemic and nonischemic conditions in patients with cardiomyopathy and in symptomatic diabetic patients undergoing ETT.

A fundamental premise tested in this study is that during rest, ECG abnormalities associated with CAD may not be evident, but when the myocardial substrate is challenged by a stimulus such as exercise, which perturbs the supply-demand relationship and increases adrenergic drive, TWH can detect occult epicardial coronary artery stenoses. Consistent with this conceptual framework, at rest, TWH_{V4-6} levels were not different in individuals with or without significant CAD. However, in response to either ETT or dipyridamole stress testing, TWH_{V4-6} was substantially increased in subjects with obstructive CAD but not in control subjects. Receiver operator characteristic curve analysis of sensitivity/specificity relationships revealed that results of TWH_{V4-6} analysis with either ETT or dipyridamole stress testing were superior to ST-segment results (FIG. 24). It is noteworthy that the AUC for TWH_{V4-6} during dipyridamole testing was superior to that during ETT (0.83 vs. 0.74, respectively).

The basis for the differences in the performance of the tests is unclear, although it is likely to be due to technical as well as conceptual considerations. An obvious distinguishing feature is that in pharmacologic testing, subjects are motionless and the movement artifacts associated with walking and running on the treadmill are avoided, potentially improving the accuracy of the ECG waveform measurements. The tests are also different from a physiologic point of view. The rationale for ETT is the imposition of a workload on the heart, which is achieved by increasing heart rate and systemic arterial pressure resulting in a challenge between supply and demand as well as an increase in cardiac sympathetic drive. The ETT test relies on patient motivation and physical capacity to exercise, as well as other factors that can impair chronotropic response such as bradycardia-inducing medications including beta-blockers and calcium channel antagonists. In contrast, dipyridamole stress testing is based on inducing inhomogeneous perfusion through steal of coronary blood flow from diseased to normal zones. It is germane in this regard that in cases, dipyridamole-induced increased TWH without a significant effect on ST-segment (Table 3). Thus, TWH is inherently suited for use with dipyridamole testing for the detection of flow-limiting coronary artery stenosis (FIG. 24).

The precise basis for TWH's capacity to detect CAD during pharmacologic stress testing with a vasodilator, when ST-segment changes have proven unreliable, is uncertain. A clue may reside in the fact that vasodilators cause only a mild metabolic challenge to the heart as a result of coronary steal from stenotic to normal regions, causing perfusion defects and wall-motion abnormalities in patients with CAD. It is well-known from the experimental and clinical literature that peaking and other changes in T-wave morphology frequently appear prior to substantial ST-segment changes. While the exact mechanisms involved have not been fully elucidated, two plausible explanations have surfaced. The first is that even mild ischemia can result in potent cardio-cardiac sympathetic reflexes, which can, through local release of norepinephrine, result in peaked T-waves in the absence of ST-segment changes. It is well-known that enhanced sympathetic activity can result in significant nonuniformities of repolarization, which can be quantified by TWH measurement, as in the present study.

A second putative mechanism is the opening of ATP-sensitive K+ channels (I_{KATP}) during myocardial ischemia through intracellular metabolic alterations including decreases in pH and ATP. Ischemia-induced activation of these channels leads to marked shortening of action potential duration (APD) such that a 1% opening of these channels has been estimated to result in a 50% shortening of APD. The fact that these channels are nonuniformly expressed in the myocardium sets the stage for marked inhomogeneity of repolarization. It is these nonuniformities that are quantified by TWH analysis and provide the underlying basis for evaluating the impact of vasodilator-induced perfusion defects.

In conclusion, TWH measurement is a novel method that can improve the diagnostic accuracy of functional stress testing with ETT and dipyridamole for detection of angiographically apparent flow-limiting stenoses in large epicardial coronary arteries.

Stress Induced Testing with Regadenoson

As noted above, myocardial perfusion imaging (MPI) with pharmacologic stress testing is the main noninvasive technique for detection of flow-limiting coronary artery disease (CAD) in patients who cannot exercise.

In a further study, it was analyzed whether incorporating the results of electrocardiographic interlead T-wave heterogeneity (TWH) with MPI improved detection of large epicardial coronary artery stenosis in a cohort of patients who underwent gold standard diagnostic coronary angiography.

The methodology used was as follows. All 59 patients at the Beth Israel Deaconess Medical Center institution who underwent stress testing with regadenoson (0.4 mg IV bolus), an A_{2A} selective agonist, within 1 month of coronary angiography from September 2017 to September 2018 were studied. Cases (N=33) had angiographically significant flow-limiting lesions (FLL) (≥50% for left main or ≥70% for other epicardial coronary arteries≥2 mm in diameter); controls (N=26) were normal or had non-FFL. TWH, i.e., interlead splay of T waves, was assessed from precordial leads V₄₋₆ by second central moment analysis by investigators blinded to results of diagnostic angiography and MPI, which was performed per standard clinical protocols.

A summary of the results are as follows. At rest, TWH_{V4-6} levels were similar for cases and controls (p=0.12). Regadenoson increased TWH_{V4-6} by 23% (p<0.0001) in cases but by 8% (p=0.08) in controls. At peak stress, TWH in cases was 45% (18 μV) higher than in controls (p=0.008). The AUC-guided optimum TWH_{V4-6} 35-μV cutpoint generated odds ratio of 24.5 (p=0.0001) and combination with MPI generated odds of 7.3 (p=0.0008).

The conclusion of the additional testing is as follows. Stress-induced TWH with regadenoson compared favorably with the diagnostic accuracy of MPI for the detection of CAD and could complement MPI as an electrocardiographic parameter for pharmacological stress testing. The details of this additional testing are provided below.

Noninvasive detection of coronary artery stenosis of large epicardial vessels remains a daily challenge in contemporary cardiology. One of the main first-line diagnostic techniques in individuals not suitable for an exercise tolerance test (ETT) is pharmacological stress testing with single-photon emission computerized tomography (SPECT) myocardial perfusion imaging (MPI) to visualize the ischemic area. In the context of SPECT testing, ST-segment depression during pharmacological stress is usually disregarded, because the electrocardiogram is not considered reliable.

Recently, a noninvasive electrocardiographic (ECG) technique has been developed to assess repolarization heterogeneity quantitatively by analyzing the second central moment of the interlead splay of T waves in precordial leads about a mean waveform as the central axis. This parameter, termed “T-wave heterogeneity” (TWH), has undergone extensive testing in the experimental laboratory, where it was shown to be highly accurate in detecting arrhythmia susceptibility during acute myocardial ischemia. In the 5600-subject Health Survey 2000 study, which was examined a representative sample of the entire Finnish population, ECG heterogeneity was found to predict cardiac mortality and sudden cardiac death with odds ratios of 3.2 to 3.5. The results have also been promising in estimating risk for arrhythmia and mortality in patients with ischemic and nonischemic cardiomyopathy and implantable cardioverter-defibrillators.

The main goal of the present study was to evaluate the capacity of TWH to detect the presence of large epicardial coronary vessel disease during pharmacologic stress testing with regadenoson, an A_{2A} selective agonist, which is in increasing use, during nuclear MPI. The study was prompted by the recognition that, during even mild myocardial ischemia, changes in ATP-sensitive potassium channel opening can significantly alter action potential duration and, in turn, T-wave morphology. It was hypothesized that heterogeneous effects on metabolism-dependent ion channels will result in nonuniformities in spatial-temporal repolarization, which can be quantified by TWH analysis, could provide a method for detecting flow-limiting epicardial coronary artery stenosis detection. The results of this novel application of TWH evaluation were compared alone and in combination with MPI for detection of CAD with reference to diagnostic coronary angiography.

Medical records from all 59 patients with readable ECGs who performed a symptom-limited regadenoson (0.4 mg IV bolus) pharmacological stress test followed by SPECT MPI and who were evaluated by diagnostic coronary angiography within 1 month of the stress test from September 2017 until September 2018 at Beth Israel Deaconess Medical Center (Boston, Mass.) were analyzed. Patients were not excluded based on clinical conditions. This study was performed under a protocol approved by the Beth Israel Deaconess Medical Center's Institutional Review Board.

Regadenoson (0.08 mg/ml; 0.4 mg IV) was infused over 20 seconds followed by a saline flush. Resting perfusion images were obtained with Tc-99m sestamibi. Tracer was injected ˜45 min prior to obtaining resting images. Following regadenoson infusion, the stress dose of sestamibi was administered IV. Stress images were obtained ˜30 min following tracer injection. Stress images were obtained ˜30 min following tracer injection. The imaging protocol involved gated SPECT. The interpretation was based on a 17-segment myocardial perfusion model by a single reader who was blinded to ECG and angiography results. Patients with reversible defects were considered positive; those with fixed or no perfusion defects were considered negative.

Cases (N=33) consisted of patients with angiographically significant flow-limiting lesions (FLL) (≥50% for left main or ≥70% for other epicardial coronary arteries≥2 mm in diameter); controls (N=26) were normal or had non-FFL.

ECGs were monitored during a baseline resting period and during pharmacological stress testing using the GE CASE 8000. Digital files of the 12-lead ECGs, sampled at 500 samples per second for each channel, were downloaded onto a secure hard drive and transferred to a second computer for second central moment analysis of TWH, i.e., the maximum splay in microvolts about the mean waveform using Mattab. Physician annotations of the time of baseline, infusion, and recovery were also input. The software program removed noise, baseline wander, and arrhythmias prior to automated estimation of T-wave heterogeneity. Since heterogeneity in T-wave morphology was measured over the entire JT interval, it did not depend on the specific T-wave endpoint as do time-dependent indices of dispersion of repolarization such as Tₚₑₐₖ−T_{end} or QT_{c} intervals.

ST-segment measurements were taken directly from the stress test final clinical report. A stress test was considered positive in patients with ST-segment depression of ≥1 mm horizontal or downsloping configuration in two contiguous leads and three consecutive beats at 80 ms after the J-point.

Statistical analyses were performed using XLSTAT (Addinsoft, Inc., New York, N.Y.). Data are reported as means±standard error of the mean (SEM). All continuous variables were normally distributed according to Shapiro-Wilk normality test. Statistical differences between cases and controls were calculated using 2-tailed unpaired Student's t-test or Welch's t-test, chosen accordingly through the application of the F-test for establishing the equality of variances. Results of TWH_{V4-6} analyses within each group were compared using paired Student's t-tests. Categorical variables were analyzed using odds ratios and Fisher's exact test. Receiver-Operating Characteristic (ROC) curves were plotted and the areas under the curve (AUC) were calculated. Differences comparing TWH_{V4-6} levels during rest and exercise were used as plot variables. Two-tailed p-values<0.05 were considered significant.

TABLE 5
Patient characteristics

| | | | Odds | | | | |
|---|---|---|---|---|---|---|---|
| Condition | Cases | Controls | ratio | 95% CI | P value | Sensitivity | Specificity |
| TWH | 31 | 10 | 24.8 | 4.8 to 127 | 0.0001 | 93.9% | 61.5% |
| MPI | 26 | 21 | 0.8 | 0.2 to 3.1 | 0.85 | 78.8% | 19.2% |
| TWH + MPI | 24 | 7 | 7.2 | 2.2 to 23 | 0.0008 | 72.7% | 73% |
| Age (average | 65 | 66 | — | — | 0.89 | — | — |
| years) | | | | | | | |
| Male | 18 | 14 | 1 | 0.4 to 2.9 | 0.95 | 54.5% | 46.1% |
| Obese (BMI >30) | 15 | 19 | 0.3 | 0.1 to 0.9 | 0.03 | 45.4% | 26.9% |
| kg/m² | | | | | | | |
| Diabetes type II | 18 | 12 | 1.4 | 0.5 to 3.9 | 0.52 | 54.5% | 53.8% |
| Hypertension | 27 | 24 | 0.4 | 0.07 to 2 | 0.25 | 81.8% | 7.7% |
| Hyperlipidemia | 28 | 18 | 2.5 | 0.7 to 8.8 | 0.15 | 84.8% | 30.7% |
| MI Hx | 21 | 6 | 5.8 | 1.8 to 18.5 | 0.0028 | 63.6% | 76.9% |
| Previous | 26 | 7 | 10 | 3 to 33.5 | 0.0002 | 78.8% | 73% |
| obstructive | | | | | | | |
| disease | | | | | | | |
| Acute coronary | 0 | 0 | 0.8 | 0.01 to 41 | 0.90 | 0% | 100% |
| syndrome | | | | | | | |
| Prior PCI | 7 | 6 | 0.9 | 0.2 to 3 | 0.86 | 21.2% | 76.9% |
| Prior CABG | 8 | 1 | 8 | 0.9 to 68.8 | 0.058 | 3% | 100% |
| Incomplete | 1 | 0 | 2.4 | 0.1 to 62.5 | 0.58 | 3% | 100% |
| revascularization | | | | | | | |
| Elevated | 13 | 2 | 7.8 | 1.5 to 38.7 | 0.012 | 39.9% | 92.3% |
| troponin (>0.01) | | | | | | | |
| Left ventricular | 10 | 7 | 0.5 | 0.1 to 2 | 0.36 | 38.4% | 46.1% |
| hypertrophy | | | | | | | |
| LVEF (average | 47% | 57% | — | — | 0.03 | — | — |
| %) | | | | | | | |
| Congestive heart | 12 | 4 | 3.1 | 0.9 to 11.3 | 0.08 | 36.4% | 84.6% |
| failure | | | | | | | |
| Chronic kidney | 7 | 3 | 2 | 0.5 to 8.9 | 0.33 | 21.2% | 88.5% |
| disease | | | | | | | |
| Bundle Branch | 2 | 2 | 0.8 | 0.1 to 5.9 | 0.80 | 6% | 92.3% |
| Block | | | | | | | |
| AF | 4 | 2 | 1.6 | 0.3 to 9.8 | 0.57 | 12.1% | 92.3% |
| Current smoker | 12 | 8 | 1.3 | 0.4 to 3.8 | 0.65 | 36.7% | 69.2% |
| Use of | 0 | 0 | 0.8 | 0.01 to 41 | 0.90 | 0% | 100% |
| pacemaker | | | | | | | |
| Beta-blocker use | 25 | 13 | 3.1 | 1 to 9.4 | 0.04 | 75.8% | 50% |
| Calcium | 8 | 8 | 0.7 | 0.2 to 2.3 | 0.57 | 24.2% | 69.2% |
| antagonist | | | | | | | |
| ACE-I or ARB | 12 | 10 | 0.9 | 0.3 to 2.6 | 0.86 | 36.3% | 61.5% |
| Statin | 26 | 15 | 2.7 | 0.9 to 8.5 | 0.08 | 78.8% | 42.3% |
| Nitrate use | 13 | 2 | 7.8 | 1.6 to 38 | 0.012 | 39.3% | 92.3% |

Key:
ACE-I: angiotensin-converting-enzyme inhibitor;
AF: atrial fibrillation;
ARB: angiotensin receptor blocker;
BMI: body mass index;
CABG: coronary artery bypass graft;
LVEF: left ventricular ejection fraction;
MI Hx: history of myocardial infarction;
MPI: myocardial perfusion imaging;
PCI: percutaneous coronary intervention;
SPECT: single-photon emission computerized tomography;
TWH: T-wave heterogeneity

The patient characteristics comparing controls with cases are provided in Table 5. The analysis found a significant difference among non-obese (BMI<30) patients (p=0.03), and those with a history of myocardial infarction (p=0.003), history of obstructive disease (p=0.0002), elevated troponin (p=0.012), and beta-blocker (p=0.04) and nitrate use (p=0.012), and left ventricular ejection fraction (LVEF) (p=0.03). However, there were no statistically significant differences among the two groups with respect to age, sex, diabetes, hypertension, hyperlipidemia, acute coronary syndrome, prior PCI, prior CABG, left ventricular hypertrophy, congestive heart failure, chronic kidney disease, bundle branch block, atrial fibrillation, current smoking, pacemaker use, calcium antagonist use, or incomplete revascularization. After multivariate analysis to account for effects of significant differences in patient characteristics, it was found that the odds ratio for TWH_{V4-6} was 22.56 (95% CI: 1.95-260.97, p=0.01).

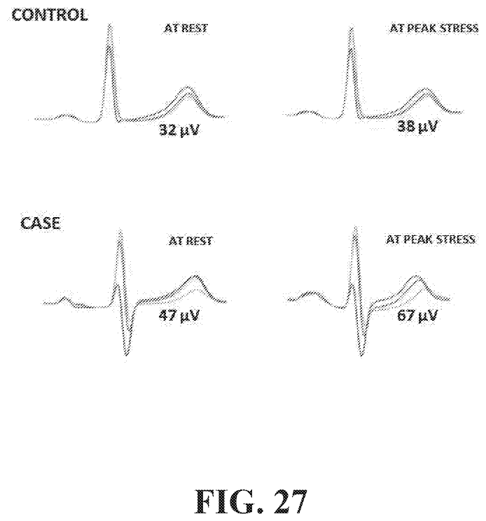

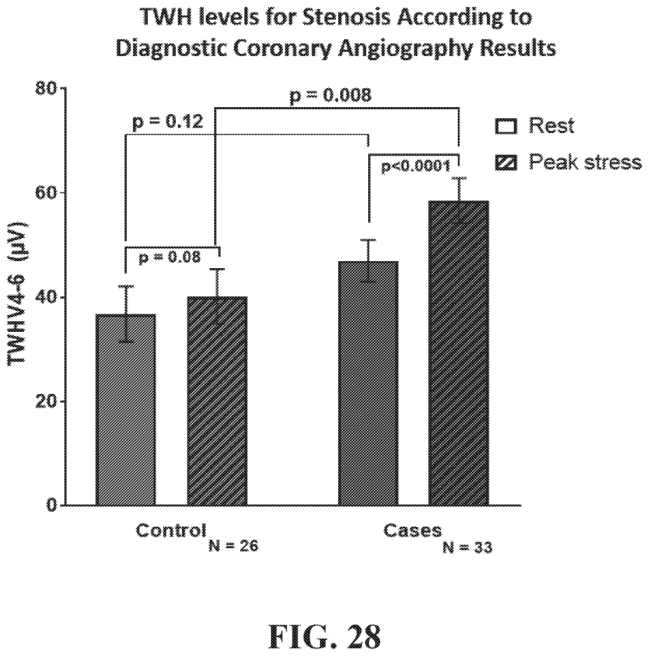

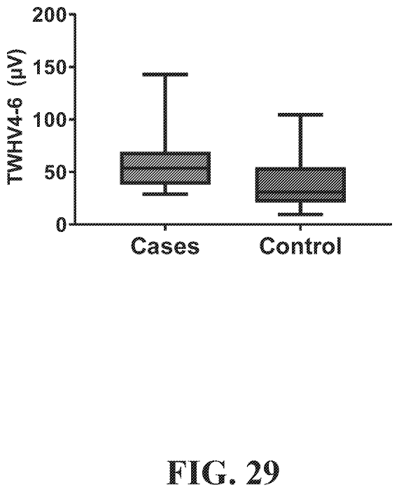

Representative examples of TWH_{V4-6} during rest and regadenoson-induced stress (FIG. 27) are provided. At rest, TWH_{V4-6} levels were similar for cases (47±4.0 μV) and controls (37±5.3 μV) (p=0.12) (FIG. 28). Regadenoson testing induced a significant 23% increase in TWH_{V4-6} in cases (to 58±4.3 μV, p<0.0001), while in controls, the 8% change was non-significant (to 40±5.2 μV, p=0.08). TWH at peak stress in cases was 45% (18 μV) higher than among controls (p=0.008). The median and 3^{rd} quartile levels of TWH_{V4-6} in cases were 71% and 31% higher, respectively, than the median and 3^{rd} quartile of TWH_{V4-6} in controls in response to regadenoson (FIG. 29).

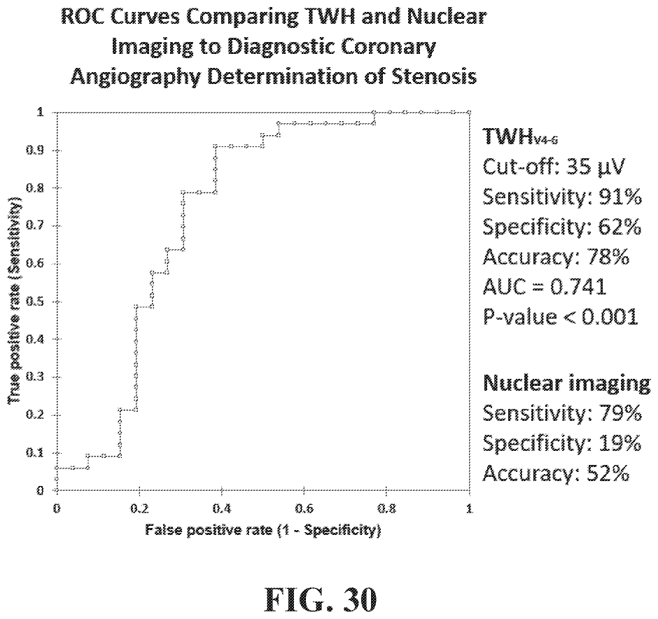

ROC analysis determined an optimal TWH_{V4-6} cutoff of 35 μV with 91% sensitivity, 61% specificity, 78% accuracy, with AUC=0.741 (p<0.001) for the presence of coronary stenosis (FIG. 30), with a significant odds ratio of 24.5 (95% CI: 4.84 to 127.04, p=0.0001). ST-segment changes did not discriminate cases from controls (p=1.0). Nuclear imaging was not significantly correlated with results of diagnostic angiography in this group (p=1.0), with 79% sensitivity, 19% specificity, and 52% accuracy and a nonsignificant odds ratio of 0.88 (95% CI: 0.24 to 3.19, p=0.88). It was investigated whether the combination of TWH_{V4-6} and nuclear imaging was correlated with diagnostic coronary angiography results. Plotted were the combination of nuclear imaging and TWH to predict coronary stenosis according to diagnostic catheterization when the result was positive only if both exams were positive and negative if either or both were negative. The odds ratio for the combination was 7.23 (95% CI: 2.27 to 23.01, p=0.0008) with 72.7% sensitivity, 73% specificity, and 72.9% accuracy.

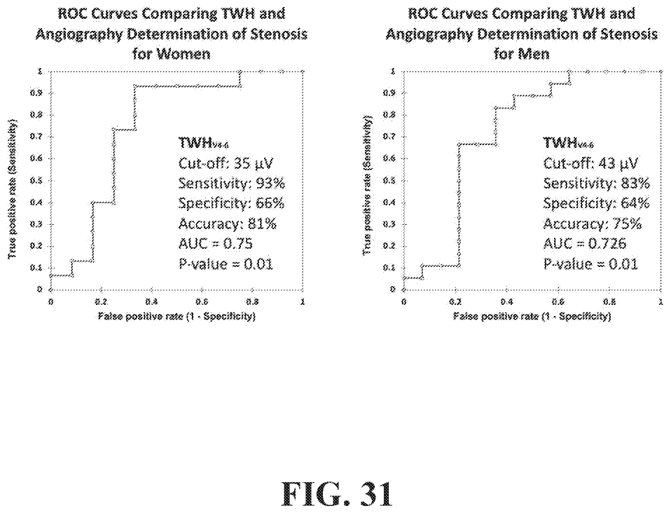

It was found that TWH_{V4-6} was similarly predictive in men and women with AUCs of 0.726 and 0.75, respectively, but that the optimized cut-off was higher in men (43 μV vs 35 μV, respectively) (FIG. 31).

This is the first study to demonstrate the potential utility of TWH_{V4-6} to detect the presence of large epicardial coronary artery stenosis in the context of regadenoson pharmacologic stress testing during MPI. To the knowledge of the inventors, no other ECG-based parameters have proved useful in this application, including ST-segment changes. The proposed concept was that because TWH is a measure of spatial nonuniformity of repolarization, even subtle changes in T-wave morphology, which antecede ST-segment changes, would be detected in the presence of mild changes in cardiac metabolism, which is known to effect energy dependent ionic gradients and consequently the uniformity of cardiac repolarization.

A sizeable body of experimental evidence supports the concept that myocardial ischemia induces marked dispersion of action potential duration and nonuniformities of recovery of excitability, which predisposes to life-threatening cardiac arrhythmias. These observations laid the groundwork for clinical investigations to determine whether repolarization heterogeneity could be employed to improve CAD detection.

The capacity of TWH to detect the presence and arrhythmogenic consequences of myocardial ischemia in response to both total and partial stenosis of large epicardial vessels including the left anterior descending and left circumflex coronary arteries has been extensively tested in large animal models. In the clinical setting, TWH has been evaluated under diverse ischemic and nonischemic conditions in patients undergoing dipyridamole stress testing, patients with cardiomyopathy and in symptomatic diabetic patients undergoing ETT.

A basic premise to be tested in the current study was whether the coronary steal effect induced by pharmacologic stress with regadenoson would induce sufficient change in cardiac metabolism and ionic shifts to result in action potential changes that would be manifest as TWH in patients with significant coronary artery stenosis. In support of this concept, it was found that there was a significant increase in TWH_{V4-6} in cases but not in control patients (FIGS. 27 and 28). Interestingly, at rest there were no differences in TWH_{V4-6} comparing control patients to cases. These results support the view that the physiologic challenge of vasodilator stress with regadenoson was an important factor in distinguishing between patients with and without stenosis assessed by diagnostic coronary angiography.

The capacity of TWH to disclose the presence of epicardial coronary stenosis is also evident in ROC curves. The area under the ROC curve (AUC) obtained with TWH during regadenoson testing was 0.74 (p<0.001). At the optimized cutpoint of 35 μV, sensitivity was 91%, specificity 62%, and accuracy 78%.

Surprisingly, results of nuclear imaging in this cohort were relatively poor, with a sensitivity of 79%, specificity of 19%, and accuracy of only 52% and a nonsignificant odds ratio of 0.8 (95% CI: 0.2 to 3.1, p=0.85). A similar picture emerged showing capacity to detect stenosis in women, when the results were differentiated based on sex. Namely, statistically significant AUC values were obtained with a 0.75 (p=0.01) for women and 0.73 (p=0.01) for men. Combining TWH with MPI increased the odds ratio of MPI from 0.8 (p=0.85) to 7.2 (p=0.0008). Theoretically, TWH could complement MPI based on the fact that it provides a continuous quantitative measurement that is not affected by factors such as image attenuation or balanced ischemia (Badheka and Hendel 2011).

Only one patient exhibited ST-segment changes during MPI testing. This individual was a female, and the ST-segment change occurred not at rest but during regadenoson administration. TWH was unchanged in response to stress. This patient was found to have a normal diagnostic angiogram and normal MPI. This case is consistent with the general problem of excessive false positive rates in women who undergo ETT based on ST-segment analysis. It was reported ST-segment changes in 25% to 66% of asymptomatic women, depending on age, which confounded the interpretation of routine treadmill exercise testing.

The exact basis for TWH's capacity to detect CAD during pharmacologic stress testing with a vasodilator, during which ST-segment alterations are unreliable, is unclear. A potential explanation is that vasodilators result in only mild metabolic stress to the heart due to coronary steal of blood flow from stenotic to normal regions. The net results are perfusion defects and wall-motion abnormalities in patients with CAD. It is well-established that peaking and other alterations in T-wave morphology usually appear prior to substantial ST-segment changes (Dressler and Roesler 1947). Whereas the precise mechanisms responsible have not been fully clarified, two main possibilities have emerged. Specifically, even mild ischemia can elicit in potent cardio-cardiac sympathetic reflexes, which can, through local release of norepinephrine, cause peaked T-waves without concurrent ST-segment changes. Increased sympathetic activity can cause significant nonuniformities of repolarization, which are quantifiable by TWH measurement, as in the current investigation.

A second plausible mechanism is the opening of ATP-sensitive K+ channels (I_{KATP}) as a result of myocardial ischemia through intracellular metabolic alterations such as decreases in pH and ATP. Ischemia-induced activation of these channels results in marked abbreviation of action potential duration (APD). It has been estimated that a 1% opening of these channels results in a 50% shortening of APD. The fact that I_{KATP} is nonuniformly expressed in the myocardium establishes conditions for marked inhomogeneity of repolarization. Accordingly, these nonuniformities can be quantified by TWH analysis and provide the scientific underpinnings for assessing the impact of vasodilator-induced perfusion defects.

Stress-induced TWH_{V4-6} with regadenoson may enhance the diagnostic accuracy of pharmacologic stress testing for detection of large epicardial coronary artery stenosis. As TWH has previously been shown to predict total and cardiac mortality and sudden death, this parameter may improve overall prognosis in the context of pharmacologic stress testing during MPI.

### Final Remarks

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method of detecting coronary artery stenosis in a patient, comprising:
receiving a first set of electrocardiogram (ECG) signals associated with the patient's heart from spatially separated leads;
generating, for each ECG signal in the first set of ECG signals, a median beat associated with the morphology of each respective ECG signal of the first set of ECG signals;
receiving a second set of ECG signals from the spatially separated leads;
generating, for each ECG signal in the second set of ECG signals a second median beat associated with the morphology of each respective ECG signal of the second set of ECG signals;
calculating, for each lead, a residuum signal based on the corresponding median beat and the corresponding second median beat;
averaging the residuum signals across the leads to produce an averaged residuum signal; and
quantifying a spatio-temporal heterogeneity of the second set of ECG signals based on the residuum signals and the averaged residuum signal, wherein the spatio-temporal heterogeneity is used to determine the presence of coronary artery stenosis.

2. The method of claim 1, wherein the spatially separated leads include leads V₁, V₂, and V₃ of a standard 12-lead ECG.

3. The method of claim 2, further comprising using the spatio-temporal heterogeneity to determine if the coronary artery stenosis exists on the right side of the patient's heart.

4. The method of claim 1, wherein the spatially separated leads include leads V₄, V₅, and V₆ of a standard 12-lead ECG.

5. The method of claim 4, further comprising using the spatio-temporal heterogeneity to determine if the coronary artery stenosis exists on the left side of the patient's heart.

6. The method of claim 1, wherein the second set of ECG signals are collected from the patient when the patient is undergoing a stress test.

7. The method of claim 6, wherein the stress test is a pharmacological stress test.

8. The method of claim 7, wherein the second set of ECG signals are collected from the patient when the patient is undergoing the pharmacological stress test through using regadenoson.

9. The method of claim 7, wherein the second set of ECG signals are collected from the patient when the patient is undergoing the pharmacological stress test through using dipyridamole.

10. The method of claim 6, wherein the stress test is an exercise tolerance test.

11. An electrocardiogram (ECG) system for detecting coronary artery stenosis in a patient, comprising:
spatially separated leads disposed around a heart of the patient and configured to measure ECG signals;
an input module configured to receive a first set of ECG signals and a second set of ECG signals from the spatially separated leads; and
a processor configured to:
generate, for each ECG signal in the first set of ECG signals, a median beat associated with the morphology of each respective ECG signal of the first set of ECG signals;
generate, for each ECG signal in the second set of ECG signals a second median beat associated with the morphology of each respective ECG signal of the second set of ECG signals;
calculate, for each lead, a residuum signal based on the corresponding median beat and the corresponding second median beat;
average the residuum signals across the leads to produce an averaged residuum signal; and
quantify a spatio-temporal heterogeneity of the second set of ECG signals based on the residuum signals and the averaged residuum signal, wherein the spatio-temporal heterogeneity is used to determine the presence of coronary artery stenosis.

12. The system of claim 11, wherein the spatially separated leads include leads V₁, V₂, and V₃ of a standard 12-lead ECG.

13. The system of claim 12, wherein the processor is further configured to use the spatio-temporal heterogeneity to determine if the coronary artery stenosis exists on the right side of the patient's heart.

14. The system of claim 11, wherein the spatially separated leads include leads V₄, V₅, and V₆ of a standard 12-lead ECG.

15. The system of claim 14, wherein the processor is further configured to use the spatio-temporal heterogeneity to determine if the coronary artery stenosis exists on the left side of the patient's heart.

16. The system of claim 11, wherein the second set of ECG signals are collected from the patient when the patient is undergoing a stress test.

17. The system of claim 16, wherein the stress test is a pharmacological stress test.

18. The system of claim 17, wherein the second set of ECG signals are collected from the patient when the patient is undergoing the pharmacological stress test through using regadenoson.

19. The system of claim 17, wherein the second set of ECG signals are collected from the patient when the patient is undergoing the pharmacological stress test through using dipyridamole.

20. The system of claim 16, wherein the stress test is an exercise tolerance test.
